(19) United States
(12) Patent Application Publication
(10) Pub. No.: US 2006/0058627 A1
(43) Pub. Date: Mar. 16, 2006

(54) Biological interface systems with wireless connection and related methods

(76) Inventors: J. Christopher Flaherty, Topsfield, MA (US); John P. Donoghue, Providence, RI (US)

Correspondence Address: Leslie I. Bookoff;FINNEGAN, HENDERSON, FARABOW,, GARRETT & DUNNER, L.L.P., 901 New York Avenue, N.W., Washington, DC, 20001-4413, US

(21) Appl. No.: 11/201,283

(22) Filed: Aug. 11, 2005

Related U.S. Application Data
(60) Provisional application No. 60/601,400, filed on Aug. 13, 2004.

(51) Int. Cl. A61B 5/05

(52) U.S. Cl. 600/409

(57) ABSTRACT

Various embodiments of a biological interface system and their related methods are disclosed. A biological interface system may include a sensor including a plurality of electrodes configured to detect multicellular signals emanating from one or more living cells of a patient and a processing unit configured to receive the multicellular signals from the sensor, to process the multicellular signals to produce processed signals, and to transmit the processed signals. The system may also include a controlled device configured to receive the processed signals from the processing unit. The processing unit may include a processing unit first portion and a processing unit second portion, where the processing unit first portion is implanted under the scalp on the skull of the patient, and the processing unit second portion is placed above the scalp of the patient at a location proximal to the processing unit first portion.

### DESCRIPTION OF THE INVENTION

This application claims the benefit of priority under 35 U.S.C. § 119(e) of U.S. provisional application No. 60/601,400, filed Aug. 13, 2004. This application relates to commonly assigned U.S. application Ser. No. ______ of Timothy R. Surgenor et al., filed on the same date as this application, and entitled “BIOLOGICAL INTERFACE SYSTEMS WITH CONTROLLED DEVICE SELECTOR AND RELATED METHODS.”

### FIELD OF THE INVENTION

The present invention relates to biological interface systems that include one or more devices controlled by processed multicellular signals of a patient. A processing unit produces a control signal based on multicellular signals received from a sensor comprising multiple electrodes. More particularly, the system includes a controlled device selector, used by the patient or other operator to select one or more devices to be controlled.

### DESCRIPTION OF RELATED ART

Biological interface devices, for example neural interface devices, are currently under development for numerous patient applications including restoration of lost function due to traumatic injury or neurological disease. Sensors, such as electrode arrays, implanted in the higher brain regions that control voluntary movement, can be activated voluntarily to generate electrical signals that can be processed by a biological interface device to create a thought invoked control signal. Such control signals can be used to control numerous devices including computers and communication devices, external prostheses, such as an artificial arm or functional electrical stimulation of paralyzed muscles, as well as robots and other remote control devices. Patients afflicted with amyotrophic lateral sclerosis (Lou Gehrig's Disease), particularly those in advanced stages of the disease, would also be applicable to receiving a neural interface device, even if just to improve communication to the external world, including Internet access, and thus improve their quality of life.

Early attempts to utilize signals directly from neurons to control an external prosthesis encountered a number of technical difficulties. The ability to identify and obtain stable electrical signals of adequate amplitude was a major issue. Another problem that has been encountered is caused by the changes that occur to the neural signals that occur over time, resulting in a degradation of system performance. Neural interface systems that utilize other neural information, such as electrocorticogram (ECOG) signals, local field potentials (LFPs) and electroencephalogram (EEG) signals have similar issues to those associated with individual neuron signals. Since all of these signals result from the activation of large groups of neurons, the specificity and resolution of the control signal that can be obtained is limited. However, if these lower resolution signals could be properly identified and the system adapt to their changes over time, simple control signals could be generated to control rudimentary devices or work in conjunction with the higher power control signals processed directly from individual neurons.

Commercialization of these neural interfaces has been extremely limited, with the majority of advances made by universities in a preclinical research setting. As the technologies advance and mature, the natural progression will be to more sophisticated human applications, such as those types of devices regulated by various governmental regulatory agencies including the Food and Drug Administration in the United States.

When sophisticated biological interface systems are commercially available it will become important for these systems to include numerous safety features required in the various locations of patient care and other patient settings. Also, systems which allow multiple devices to be controlled in a safe and reliable manner will be mandated. Convenience and flexibility to the patient, their caregivers and family members will also be a requirement.

There is therefore a need for an improved biological interface system which includes means of selecting devices to be controlled. Controlled access to the selecting means will be required. Multi-functionality, including control within the system as well as control of other devices will provide numerous benefits to the patient and the health care system.

### SUMMARY OF THE INVENTION

According to a first aspect of the invention, a biological interface system is disclosed. The biological interface system collects multicellular signals emanating from one or more living cells of a patient and transmits processed signals to a controlled device. The system comprises a sensor for detecting multicellular signals, and the sensor comprises a plurality of electrodes. The electrodes are designed to detect the multicellular signals. A processing unit is designed to receive the multicellular signals from the sensor and process the multicellular signals to produce the processed signals transmitted to the controlled device. The system includes a first controlled device for receiving the processed signals and a second controlled device for receiving the processed signals. The system further includes a selector module that is used to select the specific device to be controlled by the processed signals.

In another preferred embodiment, the biological interface system produces processed signals that include a unique identifier of the device to be controlled, and each controlled device includes means of accepting or rejecting the processed signals when the appropriate identifier is confirmed. The processed signals are preferably transmitted via wireless communication means. In an alternative embodiment, the processed signals are transmitted to one or more controlled devices with a physical connection such as wire conductors or optical fibers. Selection of the device to be controlled is accomplished with a signal selection means which determines which controlled devices receive processed signals.

In yet another preferred embodiment, the selector module includes one or more input or output elements such as visual displays, touch screens and keypads. The selector module may perform additional functions including: providing a connection to a computer network such as the Internet; reacting to a system alarm condition with an audible or visual alert, or by transmitting a distress signal to a remote site; providing a memory storage function; providing a system parameter synchronization function; providing system geographic location information; including or attaching to one or more sensors; providing a signal processing function such as to contribute to the processing unit of the biological interface system; providing a system configuration function; providing a patient feedback function such as an audible signal that correlates to one or more states of a controlled device; providing a system or patient diagnostic function; and providing a secondary function such as a personal data assistant, a phone, a cellular phone, a pager, and a calculator; an electronic game, a glucometer, a computer, a device remote control, a universal remote control, and an environmental control device.

In yet another preferred embodiment, the sensor is an array of electrodes. The electrodes may be placed into neural tissue, such as brain tissue, and one or more electrodes may stimulate tissue as well as detect cellular signals. The sensor may comprise more than one discrete component, each component including at least one electrode. The sensor components may comprise one or more of an array of electrodes, wire or wire bundle electrodes, subdural grid electrodes, scalp electrodes, and cuff electrodes.

In yet another preferred embodiment, the selection process is activated by one or more of a device, a biological signal, and an operator action. Neural and non-neural signals can be used to perform the selection. Signals generated by eye motion, eyelid motion, facial muscle, or other electromyographic activity can be used. The selection can be accomplished with devices such as: a sip and puff device; an eye gaze device; a hand, tongue or other muscle joystick or switch; another mechanical switch; an electromyogram (EMG) activated switch; and an electro-oculogram (EOG) activated switch

According to another aspect of the invention, a biological interface system is disclosed. The biological interface system collects multicellular signals emanating from one or more living cells of a patient and transmits processed signals to a controlled device. The system comprises a sensor for detecting multicellular signals, the sensor comprising a plurality of electrodes. The electrodes are designed to detect the multicellular signals. A processing unit is designed to receive the multicellular signals from the sensor and process the multicellular signals to produce the processed signals transmitted to the controlled device. The processing unit includes two components, a processing unit first portion and a processing unit second portion. The system further includes the controlled device for receiving the processed signals. The sensor is implanted within the skull of the patient, and the processing unit first portion is implanted under the scalp on the skull of the patient. The processing unit second portion is placed above the scalp of the patient at a location proximal to the processing unit first portion.

In a preferred embodiment processing unit first portion is placed in a recess in the skull, creating during a surgery, the recess at a location near but above the patient's ear. Processing unit first portion transmits neural information to processing unit second portion, through the skin, using infrared communication means. Processing unit first portion preferably does not include an embedded power supply. A coil integral to processing unit first portion converts electromagnetic signals received from processing unit second portion into power and/or data.

Both the foregoing general description and the following detailed description are exemplary and are intended to provide further explanation of the embodiments of the invention as claimed.

### BRIEF DESCRIPTION OF THE DRAWINGS

The accompanying drawings, which are incorporated in and constitute a part of this specification, illustrate various embodiments of the present invention, and, together with the description, serve to explain the principles of the invention. In the drawings:

FIG. 1 illustrates a schematic representation of the biological interface system consistent with the present invention;

FIG. 2 illustrates an exemplary embodiment of a portion of the biological system, including sensor electrodes implanted in the brain of a patient and a portion of a processing unit implanted on the skull of the patient, consistent with the present invention;

FIG. 3 illustrates another exemplary embodiment of a biological interface system consistent with the present invention wherein an operator configures the system at the patient site; and

FIG. 4 illustrates another exemplary embodiment of a biological interface system consistent with the present invention wherein a patient controls multiple devices and an operator configures the system at a site remote from the patient.

### DESCRIPTION OF THE EMBODIMENTS

To facilitate an understanding of the invention, a number of terms are defined immediately herebelow.

Definitions

As used herein, the term “biological interface system” refers to a neural interface system or any system that interfaces with living cells that produce electrical activity or cells that produce other types of detectable signals.

As used herein, the term “cellular signals” refers to subcellular signals, intracellular signals, extracellular signals, single cell signals, and signals emanating from one or more cells. “Subcellular signals” refers to: a signal derived from a part of a cell; a signal derived from one particular physical location along or within a cell; a signal from a cell extension, such as a dendrite, dendrite branch, dendrite tree, axon, axon tree, axon branch, pseudopod or growth cone; or signals from organelles, such as golgi apparatus or endoplasmic reticulum. “Intracellular signals” refers to a signal that is generated within a cell or by the entire cell that is confined to the inside of the cell up to and including the membrane. “Extracellular signals” refers to signals generated by one or more cells that occur outside of the cell(s). “Cellular signals” include but are not limited to signals or combinations of signals that emanate from any living cell. Specific examples of “cellular signals” include but are not limited to: neural signals; cardiac signals including cardiac action potentials; electromyogram (EMG) signals; glial cell signals; stomach cell signals; kidney cell signals; liver cell signals; pancreas cell signals; osteocyte cell signals; sensory organ cell signals such as signals emanating from the eye or inner ear; and tooth cell signals. “Neural signals” refers to neuron action potentials or spikes; local field potential (LFP) signals; electroencephalogram (EEG) signals; electrocorticogram signals (ECoG); and signals that are between single neuron spikes and EEG signals.

As used herein, “multicellular signals” refers to signals emanating from two or more cells, or multiple signals emanating from a single cell.

As used herein, “patient” refers to any animal, such as a mammal and preferably a human. Specific examples of “patients” include but are not limited to: individuals requiring medical assistance; healthy individuals; individuals with limited function; and in particular, individuals with lost function due to traumatic injury or neurological disease.

As used herein, “configuration” refers to any alteration, improvement, repair, calibration or other system modifying event whether manual in nature or partially or fully automated.

As used herein, “discrete component” refers to a component of a system such as those defined by a housing or other enclosed or partially enclosed structure, or those defined as being detached or detachable from another discrete component. Each discrete component can transmit information to a separate component through the use of a physical cable, including one or more of electrically conductive wires or optical fibers, or transmission of information can be accomplished wirelessly. Wireless communication can be accomplished with a transceiver that may transmit and receive data such as through the use of “Bluetooth” technology or according to any other type of wireless communication means, method, protocol or standard, including, for example, code division multiple access (CDMA), wireless application protocol (WAP), Infrared or other optical telemetry, radio frequency or other electromagnetic telemetry, ultrasonic telemetry or other telemetric technologies.

### GENERAL DESCRIPTION OF THE EMBODIMENTS

Systems and methods consistent with the invention detect cellular signals generated within a patient's body and implement various signal processing techniques to generate processed signals for transmission to one or more devices to be controlled. The system includes a sensor, comprising a plurality of electrodes that detect multicellular signals from one or more living cells, such as from the central or peripheral nervous system of a patient. The system further includes a processing unit that receives and processes the multicellular signals and transmits a processed signal to a controlled device. The processing unit utilizes various electronic, mathematic, neural net, and other signal processing techniques in producing the processed signal. Examples of controlled devices include but are not limited to prosthetic limbs, ambulation vehicles, communication devices, robots, computers, or other controllable devices.

In one exemplary embodiment, a biological interface system includes a first controlled device and a second controlled device, both controlled devices for receiving processed signals produced by the processing unit. The system further includes a selector module that is used by an operator to select the specific device to be controlled by the processed signals. Numerous configurations achieving specific device control can be implemented in the system and are described in detail herebelow. It should be noted that the selection processed as referenced in this application includes both selection of a device to be controlled by the processed signals, as well as selecting a device to stop being controlled by the processed signals. In other words, the terms “select,” “selecting,” and the “selection process” as performed by the selector module shall include both selecting and deselecting one or more controlled devices for control by the processed signals.

### DETAILED DESCRIPTION OF THE EMBODIMENTS

Reference will now be made in detail to the present embodiments of the invention, examples of which are illustrated in the accompanying drawings. Wherever possible, the same reference numbers will be used throughout the drawings to refer to the same or like parts.

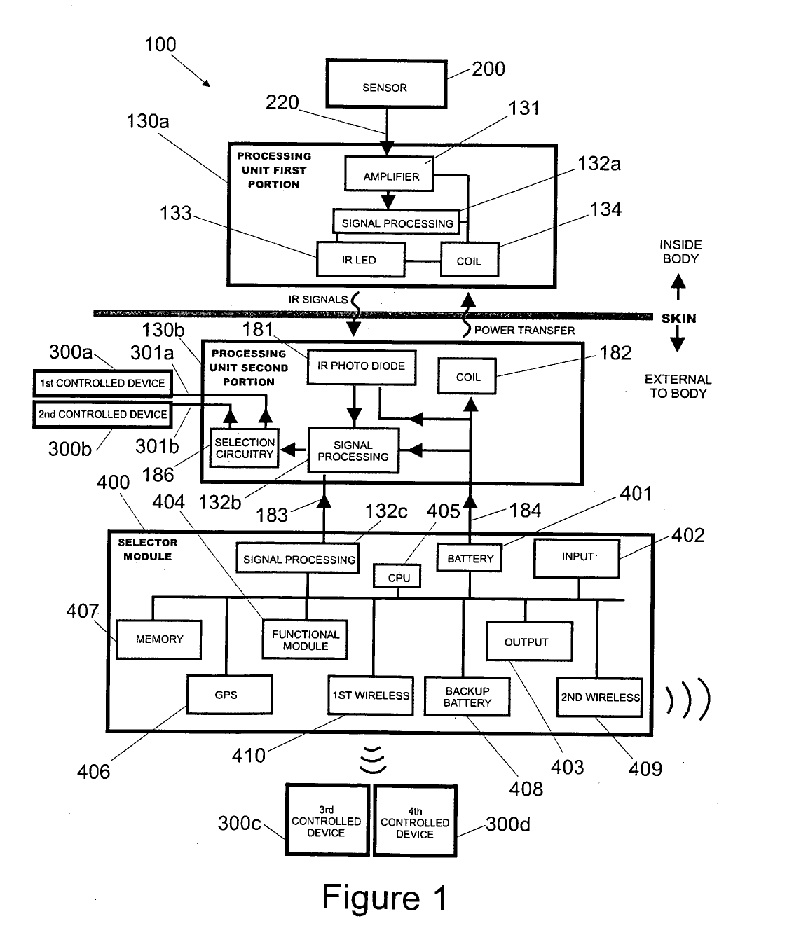

Referring now to FIG. 1, a schematic representation of a biological interface system 100 comprises implanted components and components external to the body of patient, the boundary defined schematically by a horizontal line labeled “SKIN.” A key element of system 100 is sensor 200 that includes a plurality of electrodes, not shown, for detecting multicellular signals. Sensor 200 may take various geometric forms and include numerous materials of construction, described in detail in reference to subsequent figures of this application. All exposed surfaces, such as surfaces that come in contact with tissue or bodily fluids, comprise biocompatible materials well known to those of skill in the art. In a preferred embodiment, sensor 200 includes a ten by ten matrix of electrodes; the electrodes are included at the tip of individual projections, these projections spaced at approximately 400 μm with a height of 1.0 to 1.5 mm; and the electrodes have an impedance between 100 kOhm and 1 MOhm. Sensor 200 may be placed at various locations internal and/or external to a patient, and may comprise multiple discrete components.

Another key element of system 100 is a processing unit that receives the multicellular signals from sensor 200, and utilizes one or more signal processing techniques to produce processed signals. Depicted in FIG. 1 is processing unit first portion 130a and processing unit 130b which are each a component of the processing unit of the present invention. Additional components may also be part of the processing unit, all of the components collectively performing the receiving of the multicellular signals and the production of the processed signals. Processing unit discrete components can be implanted within the patient, be external to the patient, or protrude through the skin of the patient.

As depicted in FIG. 1, processing unit first portion 130a is implanted under the skin of the patient such as on top of the skull of the patient under the scalp. In a preferred embodiment, sensor 200, also implanted, is placed within the skull such that one or more electrodes are placed within a cortical layer of the brain. Wire bundle 200, a single or multi-conductor cable, is attached to sensor 200 and processing unit first portion 130a. Wire bundle 200 attaches to one or more electrodes of sensor 200 and may include other conductors or conduits such as a conductor that provides a reference signal at a location in proximity to the electrodes of sensor 200. In a preferred embodiment, multiple individual electrodes of sensor 200 are attached each to individual conductors of wire bundle 220, and wire bundle 220 includes at least two conductors that do not attach to electrodes that are placed to provide relevant reference signals for one or more signal processing functions. In a preferred embodiment, the conductive wires of wire bundle 220 have a diameter of approximately 25 μm and comprise a blend of gold and palladium. Wire bundle 220 conductors are attached at their other end to processing unit fist portion 130a and the conductors and housing of processing unit first portion 130a are sealed such that the signals, conductive surfaces, and other internal components of wire bundle 220 and processing unit first portion 130a are appropriately protected from contamination by body fluids and other contaminants.

Processing unit first portion 130a includes means of amplifying the cellular signals, amplifier 131, which is preferably an amplifier with a gain of approximately one hundred, a working frequency range of 0.001 Hz to 7.2 kHz, a power requirement of approximately 1.6V and a power dissipation of approximately 30 mW. Processing unit first portion 130a further includes additional signal processing means, signal processing element 132a. Various signal processing techniques can be utilized including but not limited to: filtering, sorting, conditioning, translating, interpreting, encoding, decoding, combining, extracting, sampling, multiplexing, analog to digital converting, digital to analog converting, mathematically transforming, and/or otherwise processing multicellular signals to generate a control signal for transmission to a controlled device. In a preferred embodiment, signal processing element 132a includes a multiplexor function, such as a thirty-two to one multiplexor with a 1 MHz switching frequency. In another preferred embodiment, signal processing element 132a includes an analog to digital converter with twelve-bit resolution that can process 1 megasample per second for thirty-two channels.

It is desirable that all implanted components avoid the need to protrude through the skin of the patient, such as for cosmetics and reduced infection risk. In order for processing unit first portion 130a to transmit one or more signals to an external component, IR transmitter 133 is incorporated into the implant. IR transmitter 133 is preferably one or more infrared (1R) light emitting diodes (LEDs), such IR transmissions able to penetrate through a finite amount of tissue, such as the scalp. In a preferred embodiment, IR transmitter 133 transmits data at 40 megabit per second utilizing direct modulation. IR transmitter 133 receives information from signal processing element 132a, and transmits the information to processing unit second portion 130b by way of its integrated receiver, IR receiver 181. Both IR transmitter 133 and IR receiver 181 can include lenses, filters and other optical components to focus, collect, capture, or otherwise improve the IR transmission and receiving performance.

Processing unit second portion 130b, a component external to the body of the patient, is affixed or otherwise placed at a location in close proximity to the location of processing unit first portion 130a's transmitter, IR transmitter 133. In a preferred embodiment, processing unit first portion 130a is placed in a recess made in the skull, during a surgical procedure, at a location near to and above the ear of the patient. Processing unit second portion 130b is placed on the head just above the ear such that IR receiver 181 is at a location near aligned with IR transmitter 133, such as a line of site distance of approximately 4 mm. Information transfer takes place such as that using various error detection schemes, handshaking functions and other communication and error checking protocols such as ANSI X3.230 protocol and other protocols well known to those of skill of the art and applicable to digital, analog and combined digital/analog critical use communications.

Processing unit first portion 130a may include one or more additional elements, not shown, but included within, on the surface of, or attached to processing unit first portion 130a. Such elements may include but are not limited to: a temperature sensor, a pressure sensor, a strain gauge, an accelerometer, a volume sensor, an electrode, an array of electrodes, an audio transducer, a mechanical vibrator, a drug delivery device, a magnetic field generator, a photo detector element, a camera or other visualization apparatus, a wireless communication element, a light producing element, an electrical stimulator, a physiologic sensor, a heating element and a cooling element. Alternatively, processing unit first portion 130a may include an integrated power supply, not shown, to provide power to amplifier 131, signal processing element 132a, IR transmitter 133, or another component, not shown, of processing unit first portion 130a. In addition, power may be supplied to a power requiring component of sensor 200 such as by way of one or more conductors of wire bundle 220. Depicted in FIG. 1, processing unit first portion 130a includes a coil, implanted coil assembly 134, the assembly being configured to receive and convert electromagnetic signals from a device external to the body of the patient, preferably processing unit second portion 130b. Processing unit second portion 130b, also includes a coil, coil assembly 182, which is oriented within a housing of processing unit second portion 130b such that when IR Receiver 181′ is near aligned with IR Transmitter 133, coil assembly 182 can be near aligned with implanted coil assembly 134. The coil in implanted coil assembly 134 is preferably approximately 1 inch in diameter.

Through inductive coupling, power can be transferred from processing unit second portion 130b to processing unit first portion 130a by supplying a driving signal to coil assembly 182 that generates an electromagnetic field that, through inductive coupling, generates power in implanted coil assembly 134. This captured energy is converted to usable power by circuitry incorporated into implanted coil assembly 134 and can be used to power one or more elements of processing unit first portion 130a and/or recharge an integrated power supply, not shown. In the preferred embodiment shown in FIG. 1, no implanted component includes an integrated power supply such that, when coil assembly 182 is not properly energized and/or when processing unit second portion 130b is not in relative proximity to the patient, no implanted component has power. In another preferred embodiment, information can be transferred from processing unit second portion 130b to processing unit first portion 130a by modulating the waveform with circuitry included in coil assembly 182 or another component of processing unit second portion 130b. The transmission is received and decoded by the coil and circuitry of implanted coil assembly 134. This modulation pattern can easily be encoded and decoded to provide means of sending information to the implant, such as in a configuration procedure, embedding of a unique identifier, or other procedure.

Processing unit second portion 130b also includes signal processing element 132b. Signal processing can include one or more of the processes listed above in reference to signal processing element 132a and preferable includes at least a decoding function or a multiplexing function. These signal processing means, in combination with signal processing element 132a of processing unit first portion 130a may complete the processing unit function of the system of the present invention such that the two signal processing means in combination produce the processed signals that will be used to control a first controlled device, a second controlled device, or both, both not shown but described in detail in reference to subsequent figures. Processing unit second portion 130b may include wireless communication means, not shown, or wired communication means to transmit the processed signals to the controlled devices of the system. The various embodiments and elements utilizing wireless communication means can utilize radiofrequency (RF), infrared, ultrasound, microwave, other data transmission technologies that do not require a physical conductor or combinations of the preceding technologies. The various embodiments and elements utilizing wired communication means can comprise electrical conductors, optical fibers, sound wave guiding conduits, other physical cables and conductors or combinations of the preceding.

Also depicted in FIG. 1 is selector module 400, a component of the system of the present invention that is used by an operator to select one or more devices to be controlled by system 100. System 100 can have one or more operators including but not limited to: the patient; a technician; a clinician; a caregiver and a family member of the patient. In a preferred embodiment, selector module 400 can select more than one controlled device, such that processed signals control multiple controlled devices simultaneously. When multiple controlled devices are controlled simultaneously, the processed signals sent to each controlled device may be identical or different. Selector module 400 at least sends information to processing unit second portion 130b via cable 183 (e.g., a multi-conductor physical cable). It should be appreciated that various communication means could be used including but not limited to: wired connection, optical fiber connection, other physical cable communication means, wireless communication, or combinations of the preceding. At a minimum, in either wireless or physical conductor communications, processing unit second portion 130b includes data receiving means, and selector module 400 includes data transmission means, both not shown. In an alternative preferred embodiment, both processing unit second portion 130b and selector module 400 each include a transceiver element, such as a wireless transceiver element, which can both transmit and receive data.

Selector module 400 may also include signal processing means, signal processing element 132c, such that selector module 400 can perform signal processing for various purposes including contributing to the processing unit function of the system of the present invention. Signal processing can include one or more of the processes listed above in reference to signal processing element 132a. In an alternative embodiment, signal processing element 132c completes the requirements of the processing unit, in combination with signal processing element 132a of processing unit first portion 130a, and signal processing element 132b of processing unit second portion 130b, such that processed signals can be sent to the controlled devices by a data transmission element, such as information transmission means 410. In a preferred embodiment, selector module 400 performs a signal processing function, and processed signals are transmitted from selector module 400 to the controlled devices. In an alternative preferred embodiment, processing unit second portion 130b completes the signal processing of the multicellular signals, and selector module 400 transmits a selection signal to processing unit second portion 130b. This selection signal identifies which specific device is to be controlled by the processed signals.

A method of controlling one or more specific controlled devices can be accomplished by a unique identifier contained in the processed signals-transmitted to the controlled devices wherein the controlled devices includes means of identifying and/or differentiating the appropriate identifier. This identification confirming means may be a part of each controlled device, or a separate discrete-component in communication with one or more controlled devices. When a controlled device receives the proper unique identifier, control will commence. The transmission of the identifier can be at the outset of control, or may be required on a continuous basis, such as by being included with individual packets of transmitted information. A limited transmission or one-time sending of the identifier can be accompanied by an initiate command to start control. Similar approaches can be performed to cease control of one or more controlled devices. In continuous identifier transmission, cessation of control is accomplished by discontinuation of transmission of the identifier with the individual packets. In limited or one-time transmission of the identifier, the identifier can be resent and accompanied by a cessation command.

The unique controlled device identifier approach is a preferred method when processed signals are transmitted to controlled devices with wireless communication means, such that when two or more controlled devices may both be in proximity to receive the processed signals but only the appropriate one or more controlled devices will be controlled by the processed signals. An alternative method of controlling one or more specific controlled devices involves directing the processed signals to one or more specific conductors connected to one or more specific controlled devices. Referring again to FIG. 1, processing unit second portion 130b connects to first controlled device 300a with cable 301a, and processing unit second portion 130b connects to second controlled device 300b. Both cable 301a and cable 301b receive processed signals as determined by conductor selection circuitry 186. Conductor selection circuitry 186 may include solid state relays, transistor switches, or other signal switching or controlling circuitry well known to those of skill in the art. Based on the information received from selector module 400, processed signals are sent to first controlled device 300a and/or second controlled device 300b as the appropriate connections are made in conductor selection circuitry 186.

Referring again to FIG. 1, a wireless method of controlled device selection is illustrated. Selector module 400 includes an element to transmit the processed signal wirelessly, such as information transfer means 410, preferably RF wireless technology. Information transfer means 410 receives processed signals from signal processing element 132c via power and data bus 420. Power and data bus 420 is a series of conductors that include power and data signals, such as a series of conductive traces integral to a printed circuit board that connect multiple circuit board mounted components to similar conductors, such bus architecture well known to those of skill in the art. Information transfer means 410 receives power from an integrated power supply, integrated battery 401, preferably a replaceable or rechargeable battery. Numerous battery technologies, including rechargeable chemistries, can be incorporated into integrated battery 401 such as nickel cadmium or lithium iodide technologies. As depicted in FIG. 1, integrated battery 401 also provides power, via power cable 184, to processing unit second portion 130b such as to IR receiver 181, Coil Assembly 182 and signal processing element 132b. In a preferred embodiment, selector module 400 includes a redundant power supply (e.g., backup battery 408). Backup battery 408 may provide power to components of selector module 400 at specific times only such as during a power failure or during an alarm condition. In another preferred embodiment, selector module 400 attaches to a standard household outlet for access to 120VAC power through a standard plug and power cord, not shown, attached to a power converter integral to selector module 400, power converter also not shown. The power converter supplies power to the various elements of selector module 400 via bus 420 and also may recharge either or both integrated battery 401 and backup battery 408.

Information transfer means 410 transmits wireless information received by both third controlled device 300c and fourth controlled device 300d. Utilizing an embedded unique identifier transmission, and unique identifiers incorporated into third controlled device 300c and fourth controlled device 300d, each controlled device can be uniquely controlled or controlled simultaneously. The embodiment of FIG. 1 describes a system 100 that allows first controlled device 300a and second controlled device 300b to be independently controlled by processed signals received from processing unit second portion 130b as determined by inputs made to selector module 400. The system also allows third controlled device 300c and fourth controlled device 300d to be independently controlled as determined by inputs made to selector module 400, except that the processed signals are received from selector module 400. Any of the processed signals, including processed signals transmitted via a wired connection, may include the embedded unique identifier, described above, to facilitate or ensure the selection of the device to be controlled.

Selector module 400 includes a data input device, input element 402 that enables a selection of a specific controlled device to receive the processed signals of the system. Input element 402 is connected to power and data bus 420 to receive power from integrated battery 401, as are all elements attached to bus 420, and to transmit and receive signals from one or more elements of selector module 400 such as an integrated central processing unit, CPU 405 and signal processing element 132c. CPU 405 can perform numerous processing functions well known to those of skill in the art of computers and computer controlled devices. The processing functions performed by CPU 405 can work in conjunction with the various elements of selector module 400 such as those connected to bus 420. CPU 405 receives power via power and data bus 420.

Input element 402 may comprise one or more of: a keyboard, a keypad, a data entry mechanical switch or button, a mouse, a digitizing tablet, a touch screen, or other data entry element. Mechanical switches are available in various forms for persons with limited movement such as from a spinal cord injury, these patients being an applicable receiver of the system of the present invention. These forms of switches and other data entry devices include but are not limited to: a sip and puff device; an eye gaze device; a hand, tongue or other muscle joystick; an electromyogram (EMG) activated switch; and an electro-oculogram (EOG) activated switch. Input element 402 may additionally or alternatively include a voice recognition or voice activation element to select the controlled device and/or perform a different function. Alternatively or additionally, input element 402 may include a biological signal input element. Biological signals may include one or more processed signals of the system of the present invention, or a different biological signal such as one that is under voluntary control of the patient. Neural signals can be used to accomplish the selection of the device to be controlled. These neural signals may include one or more of: neuron spikes; electrocorticogram signals; local field potential signals, and electroencephalogram signals. Other signals determining the selection may include signals derived from one or more of: eye motion; eyelid motion; facial muscle; or other electromyographic activity. Signals such as EKG, respiration, and blood glucose can also be used to trigger the selection process, such as to cease control of one or more devices when an abnormal heart rate is detected. Alternatively or additionally, input element 402 may include an input that attaches to a separate device, such as a device designed for a physically impaired person. Applicable devices include but are not limited to: sip and puff devices; eye gaze devices; hand, tongue or other muscle joysticks or switches; other mechanical switches; EMG activated switches; and EOG activated switches.

Input element 402 may provide functions in addition to the selection of the controlled device to be controlled. Input element 402 may include a physical port such as a mechanical jack attached to a power line or other power receiving means such that power can be delivered to selector module 400. Wireless power receiving means may be included to allow power transfer such as through inductive coupling between mating coils. The received power may be used to power one or more elements of selector module 400 or to recharge an internal power supply such as integrated battery 401. Input element 402 may include a physical port for a different purpose, such as to provide a connection between selector module 400 and a computer network. The computer network can be one or more of: a local area network (LAN); a wide area network (WAN); a wireless fidelity network (WIFI) and the Internet. Access via a computer network such as the Internet allows selector module 400 to be accessed from a location remote to the patient of system 100 such as to retrieve information, select a controlled device or perform another function involving two-way data communication.

Input element 402 may be a mechanical switch port, such that a switch can be attached to selector module 400 to perform one or more tasks; initiate, cease or modify one or more processes or functions; or enter data. Applicable switches include but are not limited to: a sip and puff device; an eye gaze device; a hand, tongue or other muscle joystick; an electromyogram (EMG) activated switch; and an electro-oculogram (EOG) activated switch. Input element 402 may include a tilt switch, such that if selector module 400 is in an unacceptable orientation, a signal is provided via bus 420 to one or more elements. In a preferred embodiment, selector module 400 is mounted to a wheel chair, and a tilt switch would indicate when the wheelchair had fallen over. The tilt switch signal could be processed, such as by CPU 405 and selector module 400 or another component of system 100 enter an alarm condition. An audible alert can alert a nearby party, or wireless transmission of information can alert a remote party of the emergency situation. Input element 402 may include one or more sensors. A power failure sensor can be incorporated to monitor various power levels including the battery level of integrated battery 401. Other applicable sensors include but are not limited to: a physiological sensor including a neural sensor; an EKG sensor; a glucose sensor; a respiratory sensor; an activity or motion sensor; an environmental sensor; a temperature sensor; a strain gauge, an implanted sensor; a position sensor; an accelerometer; an audio sensor such as a microphone; and a visual sensor such as a photodiode.

As depicted in FIG. 1, selector module 400 includes an output element 403. In a preferred embodiment, output element 403 is used in the controlled device selection process, such as to provide output device selection means, output device information, or other system information. Output element 403 may include a visual display, such as a touch screen display, and the visual display may display selectable icons representing one or more controlled devices. Output element 403 may include a transducer, such as an audio transducer, a tactile transducer, an olfactory transducer or a visual transducer. These transducers can be used to confirm an event, such as by sounding an audible beep when a controlled device is selected or deselected, or to alert the user of an alarm or warning condition.

As depicted in FIG. 1, selector module 400 includes multiple other functional elements such as sensors, transducers, and other functional elements, input devices, and output devices. Memory storage element 407 utilizes one or more electronic memory circuitry such as RAM, ROM or other volatile and non-volatile memory storage devices. Various pieces of information can be stored including but not limited to: integrated parameter status and history of change of values; controlled device information; system change information and other historic system information; synchronization information that can be used to restore or backup information such as information that is lost due to a system or component failure, power outage, or other cause; patient information, and other information. All or part of the information stored in memory storage element 407 may also be included in a storage element of another discrete component of system 100, such as processing unit second portion 130b. In a preferred embodiment, system 100 includes a system synchronization function, such that redundant information is placed in one or more storage elements such as memory storage element 407 of selector module 400. The system synchronization function is similar to synchronization functions utilized in commercial personal data assistants (PDAs) to synchronize data between the PDA and a personal computer database of information. In system 100, the system synchronization function can place information redundantly in one or more storage modules such that if one or more components fail such as by losing a value for an integrated parameter or other system information, is replaced or otherwise is unavailable, all parameters can be reloaded utilizing the redundant data.

System 100 of FIG. 1 further includes geographic location means 406, which provides geographic position location of selector module 400 such as via a global positioning system (GPS) transducer. This geographic information can be provided to a user, such as a remote user during an alarm condition. Notification to a remote user of an alarm condition can be accomplished via an Internet connection described above, or through use of wireless communication means such as cellular telephone communications. Various alarm conditions may require assistance to the patient such as a tipped wheelchair, failed controlled device, power failure, system malfunction, undesired patient condition or other adverse events. In a preferred embodiment, system 100 includes an alarm detection element to detect one or more alarm conditions.

Selector module 400 of FIG. 1 further includes a second wireless communication element, such as redundant information transfer means 409. Information transfer means 409 provides a separate capability of communicating with a separate device such as a remote controlled device, data communication, transfer or retrieval device, or other device incorporating a wireless receiver, a wireless transmitter or a wireless transceiver. Redundant information transfer means 409 may be powered by either integrated battery 401, backup battery 408 or both. In emergency situations such as system 100 entering an alarm state, either or both information transfer means 410 and redundant information transfer means 409 may generate and/or transmit an alert or distress signal to a remote location or a remote communication device. The alert signal may include one or more of: system condition; patient condition; patient identification; system location; and patient location. Numerous events can trigger an alarm state and are described throughout this application. System 100 may enter an alarm state during one or more of: power failure; system malfunction; controlled device malfunction; controlled device in unacceptable orientation or position; and unacceptable environment encountered.

Selector module 400 further includes functional module 404, an element that can perform various functions valuable to a patient, operator or other user of system 100. The functions performed by functional module 404 may include but are not limited to: personal data assistant; phone; cellular phone; pager; calculator; electronic game; glucometer; computer; device remote control; universal remote control; and environmental control device. In a preferred embodiment, functional module 404 includes a cellular phone, and this phone can automatically dial one or more predetermined phone numbers during an alarm state or condition.

In a preferred embodiment, selector module 400 includes patient feedback means. The patient feedback means can be used to improve device control and/or to assist in patient training and system configuration. Feedback can be provided by output element 403, such as incorporating one or more of a visual display, an audible transducer, a tactile transducer or other transducer. Each transducer of output element 403 may be incorporated into or on a housing of selector module 400 or one or more transducers or displays may connect to a jack provided on selector module 400. In a preferred embodiment, the patient feedback function utilizes, at a minimum, audio feedback.

In another preferred embodiment, selector module 400 includes a separate device control function. Examples of separate devices to be controlled, such as via input element 402, include a universal remote or a medical device such as a therapeutic device, a diagnostic device, a restorative device, and an implanted device.

Selector module 400 includes one or more integrated parameters used to perform a function. These types of integrated parameters are incorporated into multiple discrete components of system 100. Examples of integrated parameters and the functions dependent on their use are described in detail throughout this application. A typical function requiring one or more integrated parameters is production of the processed signals of the present invention. The integrated parameters of selector module 400 can be stored in memory storage element 407. When the integrated parameters of selector module 400 are modified, a permission routine, described in detail in reference to a subsequent figure of this application, may be invoked.

Other functions incorporated into selector module 400 include an information retrieval function, used to retrieve current or historic information from one or more discrete components of system 100 such as selector module 400; an interrogation function used to query the current or historic status of one or more discrete components of system 100; a system diagnostic function, used to diagnose one or more conditions, occurrences or states of system 100; a patient diagnostic function, used to perform or assist in the performance of a patient diagnostic event; and a configuration function, such as a calibration or other configuration process performed on system 100 to improve system performance and safety. In a preferred embodiment, the configuration function may be performed at least one time during the use of system 100, and in another preferred embodiment, the configuration function may be successfully completed prior to initiation of control of the controlled devices of system 100.

Alternative embodiments of selector module 400 should also be considered within the spirit and scope of this application. Selector module 400 may comprise two or more discrete components, such as a wheelchair mounted component and a bed mounted component, and each discrete component may be able to operate independently with full functionality. Selector module 400 may include an embedded identifier, such as to confirm compatibility of selector module 400 with other components of system 100, the confirmation process described in detail in reference to subsequent figures. Selector module 400 may be implanted within the patient. Selector module 400 may be a controlled device of the system of the present invention.

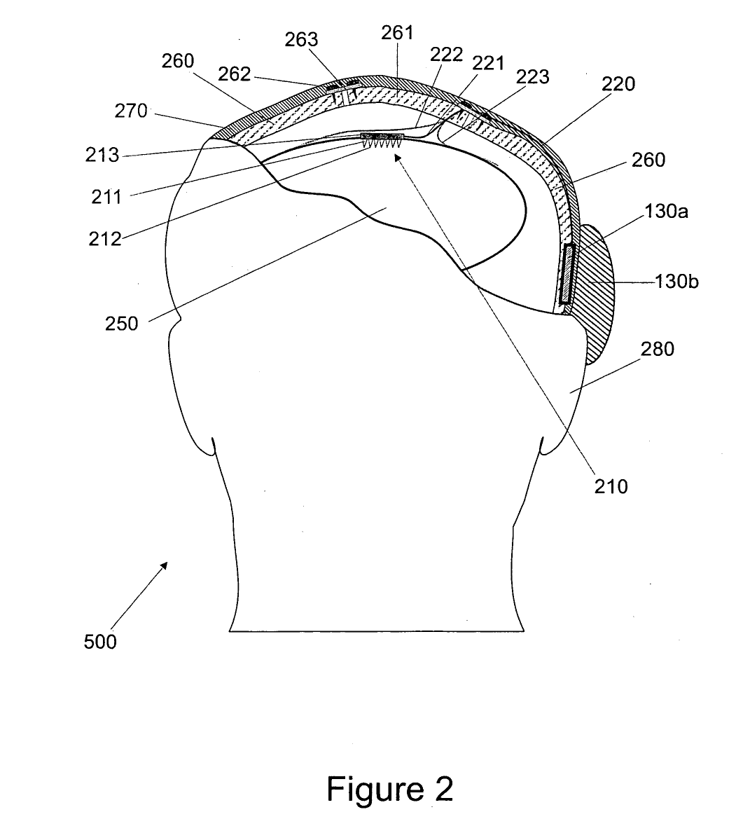

Referring now to FIG. 2, a brain implant apparatus consistent with an embodiment of the present invention is illustrated. As shown in FIG. 2, the system includes a sensor (e.g., electrode array 210) that may be inserted into a brain 250 of patient 500, through an opening surgically created in skull 260. Array 210 includes a plurality of electrodes 212 for detecting electrical brain signals or impulses. Array 210 may be placed in any location of a patient's brain allowing for electrodes 212 to detect these brain signals or impulses. In a preferred embodiment, electrodes 212 can be inserted into a part of brain 250 such as the cerebral cortex. Other locations for array 210, such as those outside of the cranium, can record cellular signals as well. Non-penetrating electrode configurations, such as subdural grids, cuff electrodes and scalp electrodes are applicable both inside the cranium such as to record local field potentials (LFPs), in, on, or near peripheral nerves, and on the surface of the scalp such as to record electroencephalogram signals (EEGs). Though FIG. 2 depicts the sensor as a single discrete component, in alternative embodiments the sensor comprises multiple discrete components. Multiple discrete components of the sensor can be implanted entirely in the brain or at an extracranial location, or the multiple discrete sensor components can be placed in any combination of locations.

Electrode array 210 serves as the sensor for the biological interface system of the present invention. While FIG. 2 shows electrode array 210 as eight electrodes 212, array 210 may include one or more electrodes having a variety of sizes, lengths, shapes, forms, and arrangements, and preferably is a ten by ten array of electrodes. Moreover, array 210 may be a linear array (e.g., a row of electrodes) or a two-dimensional array (e.g., a matrix of rows and columns of electrodes), or wire or wire bundle electrodes. An individual wire lead may include a plurality of electrodes. Electrodes may have the same materials of construction and geometry, or there may be varied materials and/or geometries used in one or more electrodes. Each electrode 212 of FIG. 2 extends into brain 250 to detect one or more cellular signals such as those generated from the neurons located in proximity to the each electrode 212's placement within the brain. Neurons may generate such signals when, for example, the brain instructs a particular limb to move in a particular way. In a preferred embodiment, the electrodes reside within the arm or leg portion of the motor cortex of the brain.

In the embodiment shown in FIG. 2, array 210 includes a sensor substrate 213 that includes multiple projections 211 emanating from a surface of the substrate 213. At the end of each projection 211 is an electrode 212. Multiple electrodes, not shown, may be included along the length of one or more of the projections 211. Projections 211 may be rigid, semi-flexible, or flexible, the flexibility of which are such that each projection 211 can still penetrate into neural tissue, potentially with an assisting device or with projections that temporarily exist in a rigid condition. One or more projections 211 may be void of any electrode, such projections potentially including anchoring means such as bulbous tips or barbs, not shown. Array 210 has previously been passed through a hole cut into skull 260, during a procedure known as a craniotomy, and inserted into brain 250 such that the projections pierce into brain 250 and sensor substrate 213 remains in close proximity to or in light contact with the surface of brain 250. The processing unit of the present invention includes processing unit first portion 130a, placed in a surgically created recess in skull 260 at a location near patient 500's ear 280. Processing unit first portion 130a receives cellular signals from array 210 via wire bundle 220, such as a multi-conductor cable. Processed signals are produced by processing unit first portion 130a and other processing unit components, such as processing unit second portion 130b located on the external skin surface of patient 500 near ear 280. The multicellular signals received from array 210 include a time code of brain activity. Processing unit first portion 130a and processing unit second portion 130b have similar elements and functionality to the identical referenced items of FIG. 1.

In the preferred embodiment depicted in FIG. 2, bone flap 261, the original bone portion removed in the craniotomy, has been used to close the hole made in the skull 260 during the craniotomy, obviating the need for a prosthetic closure implant. Bone flap 261 is attached to skull 260 with one or more straps or bands 263, that are preferably titanium or stainless steel. Band 263 is secured to bone flap 261 and skull 260 with bone screws 262. Wire bundle 220 passes between bone flap 260 and the hole cut into skull 260. During the surgical procedure, a recess was made in skull 260 such that processing unit first portion 130a could be placed in the recess, allowing scalp 270 to be relatively flat in the area proximal to processing unit first portion 130a. A long incision in the scalp between the craniotomy site and the recess can be made to place processing unit first portion 130a in the recess. Alternatively, an incision can be made to perform the craniotomy, and a separate incision made to form the recess, and the processing unit first portion 130a and wire bundle 220 can be tunneled under the scalp to the desired location. Processing unit first portion 130a is attached to skull 260 with one or more bone screws or a biocompatible adhesive, not shown.

In an alternative embodiment, processing unit first portion 130a may be placed entirely within skull 260 or be shaped and placed to fill the craniotomy hole instead of bone flap 261. Processing unit first portion 130a can be placed in close proximity to array 210, or a distance of 5-20 cm can separate the two components. Processing unit second portion 130b, placed at a location proximate to implanted processing unit first portion 130a but external to patient 500, receives information from processing unit first portion 130a via wireless communication through the skin. Processing unit second portion 130b can include means of securing to patient 500 including but not limited to: an ear attachment mechanism; a holding strap; adhesives; magnets; or other means. Processing unit second portion 130b, includes, in addition to wireless information receiving means, power transfer means, signal processing circuitry, an embedded power supply such as a battery, and information transfer means. The information transfer means of processing unit second portion 130b may include means to transfer information to one or more of: implanted processing unit first portion 130a; a different implanted device; and an external device such as an additional component of the processing unit of the present invention, a controlled device of the present invention, or a computer device such as a computer with Internet access.

Referring back to FIG. 2, electrodes 212 transfer the detected cellular signals to processing unit first portion 130a via array wires 221 and wire bundle 220. Wire bundle 220 includes multiple conductive elements, and array wires 221, which preferably include a conductor for each electrode of array 210. Also included in wire bundle 220 are two conductors, first reference wire 221 and second reference wire 222 each of which is placed in an area in relative proximity to array 210. First reference wire 221 and second reference wire 222 may be redundant and provide reference signals used by one or more signal processing elements of the processing unit of the present invention to process the cellular information detected by one or more electrodes.

Each projection 211 of electrode array 210 may include a single electrode, such as an electrode at the tip of the projection 211, or multiple electrodes along the length of each projection. Each electrode 212 may be used to detect the firing of one or more neurons, as well as other cellular signals such as those from clusters of neurons. Additional electrodes, not shown, such as those integrated into subdural grids, scalp electrodes, cuff electrodes, scalp electrodes, and other electrodes, can also detect cellular signals emanating from the central or peripheral nervous system, or other part of the body generating cellular signals, such that the processing unit uses these signals to produce the processed signals to send to the controlled device, not shown. Examples of detected signals include but are not limited to: neuron spikes, electrocorticogram signals, local field potential signals, electroencephalogram signals, and other signals between single neuron spikes and electroencephalogram signals. The processing unit may assign one or more specific cellular signals to a specific use, such as a specific use correlated to a patient imagined event. In a preferred embodiment, the one or more cellular signals assigned to a specific use are under voluntary control of the patient. In an alternative embodiment, cellular signals are transmitted to processing unit 130 via wireless technologies, such as infrared communication, such transmissions penetrating the skull of the patient, and obviating the need for wire bundle 220, array wires 221 and any physical conduit passing through skull 260 after the surgical implantation procedure is completed.

Referring back to FIG. 2, processing unit first portion 130a and processing unit second portion 130b may independently or in combination preprocess the received cellular signals (e.g., impedance matching, noise filtering, or amplifying), digitize them, and further process the cellular signals to extract neural information. Processing unit second portion 130b may then transmit the neural information to an external device (not shown), such as a further processing device and/or any device to be controlled by the processed multicellular signals. For example, the external device may decode the received neural information into control signals for controlling a prosthetic limb or limb assist device for controlling a computer cursor, or the external device may analyze the neural information for a variety of other purposes.

Processing unit first portion 130a and processing unit second portion 130b may independently or in combination also conduct adaptive processing of the received cellular signals by changing one or more parameters of the system to achieve acceptable or improved performance. Examples of adaptive processing include, but are not limited to, changing a parameter during a system configuration, changing a method of encoding neural information, changing the type, subset, or amount of neural information that is processed, or changing a method of decoding neural information. Changing an encoding method may include changing neuron spike sorting methodology, calculations, thresholds, or pattern recognition. Changing a decoding methodology may include changing variables, coefficients, algorithms, and/or filter selections. Other examples of adaptive processing may include changing over time the type or combination of types of signals processed, such as EEG, LFP, neural spikes, or other signal types.

Processing unit first portion 130a and processing unit first portion 130b may independently or in combination also transmit signals to one or more electrodes 212 such as to stimulate the neighboring nerves or other cells. Stimulating electrodes in various locations can be used by processing unit 130 to transmit signals to the central nervous system, peripheral nervous system, other body systems, body organs, muscles, and other tissue or cells. The transmission of these signals is used to perform one or more functions including but not limited to: pain therapy, muscle stimulation, seizure disruption, and patient feedback.

Processing unit first portion 130a and processing unit second portion 130b independently or in combination include signal processing circuitry to perform one or more functions including but not limited to: amplification, filtering, sorting, conditioning, translating, interpreting, encoding, decoding, combining, extracting, sampling, multiplexing, analog to digital converting, digital to analog converting, mathematically transforming, and otherwise processing cellular signals to generate a control signal for transmission to a controlled device. Processing unit first portion 130a transmits raw or processed cellular information to processing unit second portion 130b through integrated wireless communication means, such as radiofrequency communications, infrared communications, inductive communications, ultrasound communications, and microwave communications. This wireless transfer allows the array 210 and processing unit first portion 130a to be completely implanted under the skin of the patient, avoiding the need for implanted devices that require protrusion of a portion of the device through the skin surface. Processing unit first portion 130a may further include a coil, not shown, which can receive power, such as through inductive coupling, on a continual or intermittent basis from an external power transmitting device as has been described in detail in reference to FIG. 1. In addition to or in place of power transmission, this integrated coil and its associated circuitry may receive information from an external coil whose signal is modulated in correlation to a specific information signal. The power and information can be delivered to processing unit first portion 130a simultaneously such as through simple modulation schemes in the power transfer that are decoded into information for processing unit first portion 130 to use, store, or facilitate another function. A second information transfer means, in addition to a wireless means such as an infrared led, can be accomplished by modulating a signal in the coil of processing unit first portion 130a that information is transmitted from the implant to an external device including a coil and decoding elements.

In an alternative embodiment, not shown, processing unit first portion 130a, and potentially additional signal processing functions are integrated into array 210, such as through the use of a bonded electronic microchip. In another alternative embodiment, processing unit first portion 130a may also receive non-neural cellular signals and/or other biologic signals, such as from an implanted sensor. These signals may be in addition to received neural multicellular signals, and they may include but are not limited to: EKG signals, respiration signals, blood pressure signals, electromyographic activity signals, and glucose level signals. Such biological signals may be used to turn the biological interface system of the present invention, or one of its discrete components, on or off, to begin a configuration routine, or to start or stop another system function. In another alternative embodiment, processing unit first portion 130a and processing unit second portion 130b independently or in combination produce one or more additional processed signals, to additionally control the controlled device of the present invention or to control one or more additional controlled devices.

In an alternative embodiment, a discrete component such as a sensor of the present invention, is implanted within the cranium of the patient, such as array 210 of FIG. 2, a processing unit, or a portion of a processing unit of the present invention is implanted in the torso of the patient, and one or more discrete components are external to the body of the patient. The processing unit may receive multicellular signals from the sensor via wired communication, including conductive wires and optic fibers, or wireless communication.

Each sensor discrete component of the present invention can have as few as a single electrode, with the sensor including multiple sensor discrete components that collectively contain a plurality of electrodes. Each electrode is capable of recording a plurality of neurons, or other electrical activity. In an alternative embodiment, one or more electrodes are included in the sensor to deliver electrical signals or other energy to the tissue neighboring the electrode, such as to stimulate, polarize, hyperpolarize, or otherwise cause an effect on one or more cells of neighboring tissue. Specific electrodes may record cellular signals only, or deliver energy only, and specific electrodes may provide both functions.

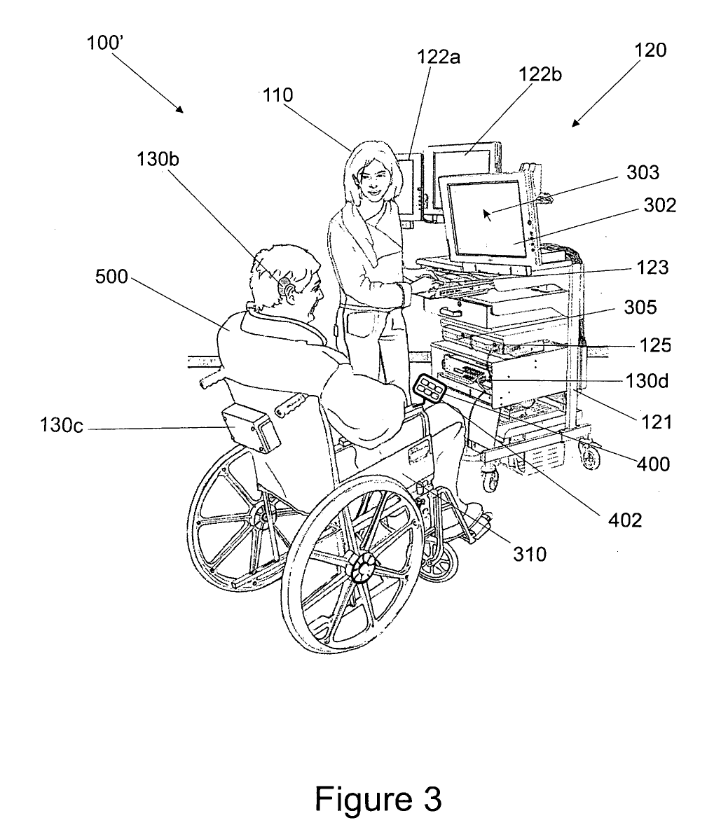

Referring now to FIG. 3, a biological interface system 100′ comprises implanted components, not shown, and components external to the body of a patient 500. A sensor for detecting multicellular signals, preferably a two dimensional array of multiple protruding electrodes, may be implanted in the brain of patient 500 in an area such as the motor cortex. In a preferred embodiment, the sensor is placed in an area to record multicellular signals that are under voluntary control of the patient. Alternatively or additionally to the two dimensional array, the sensor may include one or more wires or wire bundles which include a plurality of electrodes. Patient 500 of FIG. 3 is shown as a human being, but other mammals and life forms that produce recordable multicellular signals would also be applicable. Patient 500 may be a patient with a spinal cord injury or afflicted with a neurological disease that has resulted in a loss of voluntary control of various muscles within the patient's body. Alternatively or additionally, patient 500 may have lost a limb, and system 100′ will include a prosthetic limb as its controlled device.

The sensor electrodes of system 100′ can be used to detect various multicellular signals including neuron spikes, electrocorticogram signals (ECoG), local field potential (LFP) signals, electroencelphalogram (EEG) signals, and other cellular and multicellular signals. The electrodes can detect multicellular signals from clusters of neurons and provide signals midway between single neuron and electroencephalogram recordings. Each electrode is capable of recording a combination of signals, including a plurality of neuron spikes. The sensor can be placed on the surface of the brain without penetrating, such as to detect local field potential (LFP) signals, or on the scalp to detect electroencephalogram (EEG) signals.

A portion of the processing unit, such as processing unit second portion 130b receives signals from an implanted processing unit component, such as has been described in reference to FIG. 1 and FIG. 2. Processing unit second portion 130b is located just above the ear of patient 500, such that the data transmitting implanted component is located under the scalp in close proximity to the location of processing unit second portion 130b, as depicted in FIG. 3. Signals are transmitted from the implanted processing unit component to processing unit second portion 130b using wireless transmission means. The processing unit components of system 100′ perform various signal processing functions including but not limited to: amplification, filtering, sorting, conditioning, translating, interpreting, encoding, decoding, combining, extracting, sampling, multiplexing, analog to digital converting, digital to analog converting, mathematically transforming, and/or otherwise processing cellular signals to generate a control signal for transmission to a controllable device. The processing unit may process signals that are mathematically combined, such as the combining of neuron spikes that are first separated using spike discrimination methods, these methods known to those of skill in the art. In alternative embodiments, the processing unit may comprise multiple components or a single component, and each of these processing unit components can be fully implanted in patient 500, be external to the body, or be implanted with a portion of the component exiting through the skin.

In FIG. 3, one controlled device is a computer, such as CPU 305 which is attached to monitor 302. Through the use of system 100′, patient 500 can control cursor 303 of CPU 305 and potentially other functions of the computer such as turning it on and off, keyboard entry, joystick control, or control of another input device, each function individually or in combination. System 100′ includes another controlled device, such as wheelchair 310. Numerous other controlled devices can be included in the systems of this application, individually or in combination, including but not limited to: a computer; a computer display; a mouse; a cursor; a joystick; a personal data assistant; a robot or robotic component; a computer controlled device; a teleoperated device; a communication device or system; a vehicle such as a wheelchair; an adjustable bed; an adjustable chair; a remote controlled device; a Functional Electrical Stimulator device or system; a muscle stimulator; an exoskeletal robot brace; an artificial or prosthetic limb; a vision enhancing device; a vision restoring device; a hearing enhancing device; a hearing restoring device; a movement assist device; medical therapeutic equipment such as a drug delivery apparatus; medical diagnostic equipment such as epilepsy monitoring apparatus; other medical equipment such as a bladder control device, a bowel control device and a human enhancement device; closed loop medical equipment and other controllable devices applicable to patients with some form of paralysis or diminished function as well as any device that may be utilized under direct brain or thought control in either a healthy or unhealthy patient.

The sensor is connected via a multi-conductor cable implanted in patient 500 to an implanted portion of the processing unit which includes some signal processing elements as well as wireless communication means as has been described in detail in reference to FIG. 1 and FIG. 2. The implanted multi-conductor cable preferably includes a separate conductor for each electrode, as well as additional conductors to serve other purposes, such as providing reference signals and ground.

Processing unit second portion 130b includes various signal processing elements including but not limited to: amplification, filtering, sorting, conditioning, translating, interpreting, encoding, decoding, combining, extracting, sampling, multiplexing, analog to digital converting, digital to analog converting, mathematically transforming, and/or otherwise processing cellular signals to generate a control signal for transmission to a controllable device. Processing unit second portion 130b includes a unique electronic identifier, such as a unique serial number or any alphanumeric or other retrievable, identifiable code associated uniquely with the system 100′ of patient 500. The unique electronic identifier may take many different forms in processing unit second portion 130b, such as a piece of electronic information stored in a memory module; a semiconductor element or chip that can be read electronically via serial, parallel, or telemetric communication; pins or other conductive parts that can be shorted or otherwise connected to each other or to a controlled impedance, voltage or ground, to create a unique code; pins or other parts that can be masked to create a binary or serial code; combinations of different impedances used to create a serial code that can be read or measured from contacts, features that can be optically scanned and read by patterns and/or colors; mechanical patterns that can be read by mechanical or electrical detection means or by mechanical fit, a radio frequency identifier or other frequency spectral codes sensed by radiofrequency or electromagnetic fields, pads and/or other marking features that may be masked to be included or excluded to represent a serial code, or any other digital or analog code that can be retrieved from the discrete component.

Alternatively or in addition to embedding the unique electronic identifier in processing unit second portion 130b, the unique electronic identifier can be embedded in one or more implanted discrete components. Under certain circumstances, processing unit second portion 130b or another external or implanted component may need to be replaced, temporarily or permanently. Under these circumstances, a system compatibility check between the new component and the remaining system components can be confirmed at the time of the repair or replacement surgery through the use of the embedded unique electronic identifier.

The unique electronic identifier can be embedded in one or more of the discrete components at the time of manufacture, or at a later date such as at the time of any clinical procedure involving the system, such as a surgery to implant the sensor electrodes into the brain of patient 500. Alternatively, the unique electronic identifier may be embedded in one or more of the discrete components at an even later date such as during a system configuration such as a calibration procedure.

Referring again to FIG. 3, processing unit second portion 130b communicates with one or more discrete components of system 100′ via wireless communication means. Processing unit second portion 130b communicates with selector module 400, a component utilized to select the specific device to be controlled by the processed signals of system 100′. Selector module 400 includes an input element 402, such as a set of buttons, used to perform the selection process. The functionality of selector module 400 has been described in detail in reference to FIG. 1. Processing unit second portion 130b also communicates with controlled device CPU 305, such as to control cursor 303 or another function of CPU 305. Processing unit second portion 130b also communicates with processing unit third portion 130c. Processing unit third portion 130c provides additional signal processing functions, as have been described above, to control wheelchair 310. System 100′ of FIG. 3 utilizes selector module 400 to select one or more of CPU 305, wheelchair 310, or another controlled device, not shown, to be controlled by the processed signals produced by the processing unit of the present invention. System 100′ also includes a modality wherein one set of processed signals emanate from one portion of the processing unit, such as processing unit second portion 130b, and a different set of processed signals emanate from a different portion of the processing unit, such as processing unit third portion 130c.

The various components of system 100′ communicate with wireless transmission means, however it should be appreciated that physical cables can be used to transfer information alternatively or in addition to wireless means. These physical cables may include electrical wires, optical fibers, sound wave guide conduits, and other physical means of transmitting data and/or power, and any combination of those means.

A qualified individual, such as operator 110, may perform a configuration of system 100′ at some time during the use of system 100, preferably soon after implantation of the sensor. In a preferred embodiment, at least one configuration routine is performed and successfully completed by operator 110 prior to use of system 100′ by patient 500. As depicted in FIG. 3, operator 110 utilizes configuration apparatus 120 which includes first configuration monitor 122a, second configuration monitor 122b, configuration keyboard 123, and configuration CPU 125, to perform a calibration routine or other system configuration process such as patient training, algorithm and algorithm parameter selection, and output device setup. The software programs and hardware required to perform the configuration can be included in the processing unit, such as processing unit second portion 130b, be included in selector module 400, or be incorporated into configuration apparatus 120. Configuration apparatus 120 may include additional input devices, such as a mouse or joystick, not shown. Configuration apparatus 120 may include various elements, functio ns and data including but not limited to: memory storage for future recall of configuration activities, operator qualification routines, standard human data, standard synthesized or artificial data, neuron spike discrimination software, operator security and access control, controlled device data, wireless communication means, remote (such as via the Internet) configuration communication means, and other elements, functions, and data used to provide an effective and efficient configuration on a broad base of applicable patients and a broad base of applicable controlled devices. The unique electronic identifier can be embedded in one or more of the discrete components at the time of system configuration, including the act of identifying a code that was embedded into a particular discrete component at its time of manufacture, and embedding that code in a different discrete component. In an alternative embodiment, all or part of the functionality of configuration apparatus 120 is integrated into selector module 400 such that system 100′ can perform one or more configuration processes such as a calibration procedure utilizing selector module 400 without the availability of configuration apparatus 120.

In a preferred embodiment, an automatic or semi-automatic configuration function or routine is embedded in system 100′. This embedded configuration routine can be used in place of a configuration routine performed manually by operator 110 as is described hereabove, or can be used in conjunction with one or more manual configurations. Automatic and/or semi-automatic configuration events can take many forms including but not limited to: monitoring of cellular activity, wherein the system automatically changes which particular signals are chosen to produce the processed signals; running parallel algorithms in the background of the one or more algorithms currently used to create the processed signals, and changing one or more algorithms when improved performance is identified in the background event; monitoring of one or more system functions, such as alarm or warning condition events or frequency of events, wherein the automated system shuts down one or more functions and/or improves performance by changing a relevant variable; and other methods that monitor one or more pieces of system data, identify an issue or potential improvement, and determine new parameters that would reduce the issue or achieve an improvement. In a preferred embodiment of the disclosed invention, when specific integrated parameters are identified, by an automated or semi-automated calibration or other configuration routine, to be modified for the reasons described above, an integral permission routine of the system requires approval of a specific operator when one or more of the integrated parameters is modified.

Operator 110 may be a clinician, technician, caregiver, patient family member, or even the patient themselves in some circumstances. Multiple operators may be needed or required to perform a configuration or approve a modification of an integrated parameter, and each operator may be limited by system 100′, via passwords and other control configurations, to only perform or access specific functions. For example, only the clinician may be able to change specific critical parameters, or set upper and lower limits on other parameters, while a caregiver, or the patient, may not be able to access those portions of the configuration procedure or the permission procedure. The configuration procedure includes the setting of numerous parameters needed by system 100′ to properly control one or more controlled devices. The parameters include but are not limited to various signal conditioning parameters as well as selection and de-selection of specific multicellular signals for processing to generate the device control creating a subset of signals received from the sensor to be processed. The various signal conditioning parameters include, but are not limited to, threshold levels for amplitude sorting, other sorting and pattern recognition parameters, amplification parameters, filter parameters, signal conditioning parameters, signal translating parameters, signal interpreting parameters, signal encoding and decoding parameters, signal combining parameters, signal extracting parameters, mathematical parameters including transformation coefficients, and other signal processing parameters used to generate a control signal for transmission to a controlled device.

The configuration routine will result in the setting of various configuration output parameters, all such parameters to be considered integrated parameters of the system of the present invention. Configuration output parameters may comprise but are not limited to: electrode selection, cellular signal selection, neuron spike selection, electrocorticogram signal selection, local field potential signal selection, electroencephalogram signal selection, sampling rate by signal, sampling rate by group of signals, amplification by signal, amplification by group of signals, filter parameters by signal, and filter parameters by group of signals. In a preferred embodiment, the configuration output parameters are stored in memory in one or more discrete components, and the parameters are linked to the system's unique electronic identifier.

Calibration and other configuration routines, including manual, automatic, and semi-automatic routines, may be performed on a periodic basis, and may include the selection and deselection of specific cellular signals over time. The initial configuration routine may include initial values, or starting points, for one or more of the configuration output parameters. Setting initial values of specific parameters, may invoke a permission routine. Subsequent configuration routines may involve utilizing previous configuration output parameters that have been stored in a memory storage element of system 100′. Subsequent configuration routines may be shorter in duration than an initial configuration and may require less patient involvement. Subsequent configuration routine results may be compared to previous configuration results, and system 100′ may require a repeat of configuration if certain comparative performance is not achieved.

The configuration routine may include the steps of (a) setting a preliminary set of configuration output parameters; (b) generating processed signals to control the controlled device; (c) measuring the performance of the controlled device control; and (d) modifying the configuration output parameters. The configuration routine may further include the steps of repeating steps (b) through (d). The configuration routine may also require invoking the permission routine of the present invention.

In the performance of the configuration routine, the operator 110 may involve patient 500 or perform steps that do not involve the patient. The operator 110 may have patient 500 imagine one or more particular movements, imagined states, or other imagined events, such as a memory, an emotion, the thought of being hot or cold, or other imagined event not necessarily associated with movement. The patient participation may include the use of one or more cues such as audio cues, visual cues, olfactory cues, and tactile cues. The patient 500 may be asked to imagine multiple movements, and the output parameters selected during each movement may be compared to determine an optimal set of output parameters. The imagined movements may include the movement of a part of the body, such as a limb, arm, wrist, finger, shoulder, neck, leg, angle, and toe, and imagining moving to a location, moving at a velocity or moving at an acceleration. The patient may imagine the movement while viewing a video or animation of a person performing the specific movement pattern. In a preferred embodiment, this visual feedback is shown from the patient's perspective, such as a video taken from the person performing the motion's own eye level and directional view. Multiple motion patterns and multiple corresponding videos may be available to improve or otherwise enhance the configuration process. The configuration routine correlates the selected movement with modulations in the multicellular signals received from the sensor, such as by correlating the periodicity of the movement with a periodicity found in one or more cellular signals. Correlations can be based on numerous variables of the motion including but not limited to position, velocity, and acceleration.

The configuration routine will utilize one or more configuration input parameters to determine the configuration output parameters. In addition to the multicellular signals themselves, system or controlled device performance criteria can be utilized. Other configuration input parameters include various properties associated with the multicellular signals including one or more of: signal to noise ratio, frequency of signal, amplitude of signal, neuron firing rate, average neuron firing rate, standard deviation in neuron firing rate, modulation of neuron firing rate as well as a mathematical analysis of any signal property including but not limited to modulation of any signal property. Additional configuration input parameters include but are not limited to: system performance criteria, controlled device electrical time constants, controlled device mechanical time constants, other controlled device criteria, types of electrodes, number of electrodes, patient activity during configuration, target number of signals required, patient disease state, patient condition, patient age, and other patient parameters and event based (such as a patient imagined movement event) variations in signal properties including neuron firing rate activity. In a preferred embodiment, one or more configuration input parameters are stored in memory and linked to the embedded, specific, unique electronic identifier. All configuration input parameters shall be considered an integrated parameter of the system of the present invention.

It may be desirous for the configuration routine to exclude one or more multicellular signals based on a desire to avoid signals that respond to certain patient active functions, such as non-paralyzed functions, or even certain imagined states. The configuration routine may include having the patient imagine a particular movement or state, and based on sufficient signal activity such as firing rate or modulation of firing rate, exclude that signal from the signal processing based on that particular undesired imagined movement or imagined state. Alternatively, real movement accomplished by the patient may also be utilized to exclude certain multicellular signals emanating from specific electrodes of the sensor. In a preferred embodiment, an automated or semi-automated calibration or other configuration routine may include through addition, or exclude through deletion, a signal based on insufficient activity during known patient movements.

Patient 500 of FIG. 3 can be a quadriplegic, a paraplegic, an amputee, a spinal cord injury victim, or a physically impaired person. Alternatively or in addition, patient 500 may have been diagnosed with one or more of: obesity, an eating disorder, a neurological disorder, a psychiatric disorder, a cardiovascular disorder, an endocrine disorder, sexual dysfunction, incontinence, a hearing disorder, a visual disorder, sleeping disorder, a movement disorder, a speech disorder, physical injury, migraine headaches, or chronic pain. System 100′ can be used to treat one or more medical conditions of patient 500, or to restore, partially restore, replace, or partially replace a lost function of patient 500.

Alternatively, system 100 can be utilized by patient 500 to enhance performance, such as if patient 500 did not have a disease or condition from which a therapy or restorative device could provide benefit, but did have an occupation wherein thought control of a device provided an otherwise unachieved advancement in healthcare, crisis management, and national defense. Thought control of a device can be advantageous in numerous healthy individuals including but not limited to: a surgeon, such as an individual surgeon using thought control to maneuver three or more robotic arms in a complex laparoscopic procedure; a crisis control expert, such as a person who in attempting to minimize death and injury uses thought control to communicate different pieces of information and/or control multiple pieces of equipment, such as urban search and rescue equipment, simultaneously during an event such as an earthquake or other disaster, both natural disasters and those caused by man; a member of a bomb squad, such as an expert who uses thoughts to control multiple robots and/or robotic arms to remotely diffuse a bomb; and military personnel who use thought control to communicate with personnel and control multiple pieces of defense equipment, such as artillery, aircraft, watercraft, land vehicles, and reconnaissance robots. It should be noted that the above advantages of system 100′ to a healthy individual are also advantages achieved in a patient such as a quadriplegic or paraplegic. In other words, a quadriplegic could provide significant benefit to society, such as in controlling multiple bomb diffusing robots, in addition to his or her own ambulation and other quality of life devices. Patients undergoing implantation and use of the system 100′ of the present invention may provide numerous occupational and other functions not available to individuals that do not have the biological interface system of the present invention.

The systems of the present invention, such as system 100′ of FIG. 3, include a processing unit that processes multicellular signals received from patient 500. Processing unit second portion 130b and other processing unit components, singly or in combination, perform one or more functions. The functions performed by the processing unit include but are not limited to: producing the processed signals; transferring information to a separate device; receiving information from a separate device; producing processed signals for a second controlled device; activating an alarm, alert or warning; shutting down a part of or the entire system; ceasing control of a controlled device; storing information, and performing a configuration.

In order for the processing unit of system 100′ to perform one or more functions, one or more integrated parameters are utilized. These parameters include pieces of information stored in, sent to, or received from, any component of system 100, including but not limited to: the sensor; a processing unit component; processing unit second portion 130b; or a controlled device. Parameters can be received from devices outside of system 100′ as well, such as configuration apparatus 120, a separate medical therapeutic or diagnostic device, a separate Internet based device, or a separate wireless device. These parameters can be numeric or alphanumeric information, and can change over time, either automatically or through an operator involved configuration or other procedure.

In order to change an integrated parameter, system 100′ includes a permission routine, such as an embedded software routine or software driven interface that allows the operator to view information and enter data into one or more components of system 100. The data entered must signify an approval of the parameter modification in order for the modification to take place. Alternatively, the permission routine may be partially or fully located in a separate device such as configuration apparatus 120 of FIG. 3, or a remote computer such as a computer that accesses system 100′ via the Internet or utilizing wireless technologies. In order to access the permission routine and/or approve the modification of the integrated parameters, a password or security key, either mechanical, electrical, electromechanical, or software based, may be required of the operator. Multiple operators may be needed or required to approve a parameter modification. Each specific operator or operator type may be limited by system 100′, via passwords and other control configurations, to approve the modification of only a portion of the total set of modifiable parameters of the system. Additionally or alternatively, a specific operator or operator type may be limited to only approve a modification to a parameter within a specific range of values, such as a range of values set by a clinician when the operator is a family member. Operator or operator types, hereinafter operator, include but are not limited to: a clinician, primary care clinician, surgeon, hospital technician, system 100′ supplier or manufacturer technician, computer technician, family member, immediate family member, caregiver, and patient.

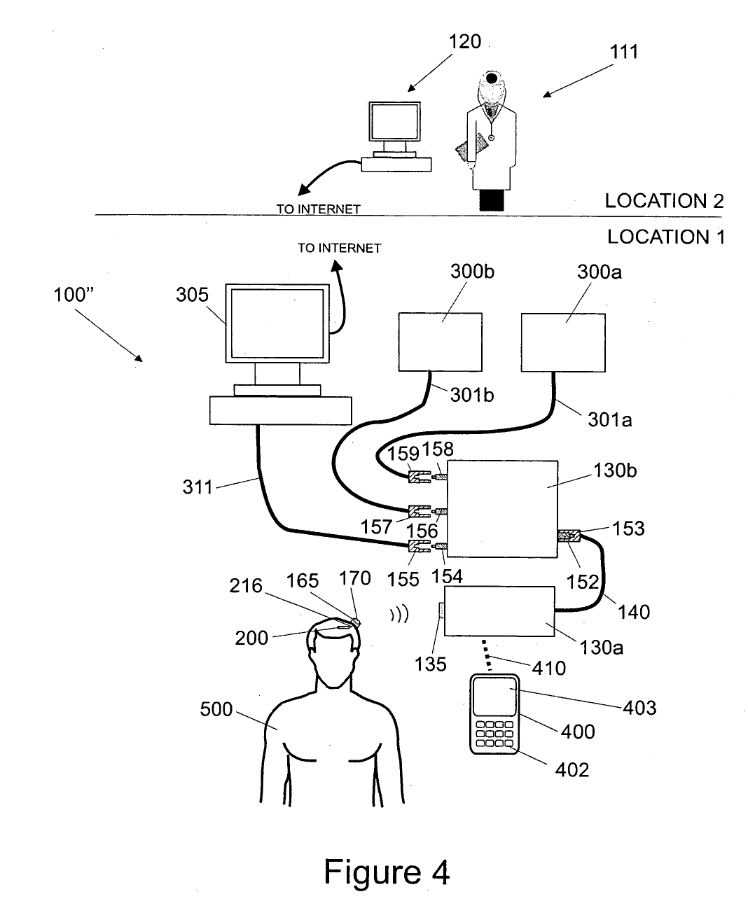

Referring now to FIG. 4, a biological interface system 100″ comprises implanted components and components external to the body of patient 500. System 100″ includes multiple controlled devices, such as controlled computer 305, first controlled device 300a, and second controlled device 300b. While three controlled devices are depicted, this particular embodiment includes any configuration of two or more controlled devices for a single patient. First controlled device 300a and second controlled device 300b can include various types of devices such as prosthetic limbs or limb assist devices, robots or robotic devices, communication devices, computers, and other controllable devices as have been described in more detail hereabove. The multiple controlled devices can include two or more joysticks or simulated joystick interfaces, two or more computers, a robot and another controlled device, and many other combinations and multiples of devices as have been described in detail hereabove. Each controlled device includes one or more discrete components or is a portion of a discrete component.

A sensor 200 for detecting multicellular signals, preferably a two dimensional array of multiple protruding electrodes, has been implanted in the brain of patient 500 in an area such as the motor cortex. In a preferred embodiment, the sensor 200 is placed in an area to record multicellular signals that are under voluntary control of the patient. Alternatively or additionally to the two dimensional array, the sensor may include: an additional array; one or more wires or wire bundles which include a plurality of electrodes; subdural grids; cuff electrodes; scalp electrodes; or other single or multiple electrode configurations. Sensor 200 is attached to transcutaneous connector 165 via wiring 216, a multi-conductor cable that preferably, though not necessarily, includes a separate conductor for each electrode of sensor 200. Transcutaneous connector 165 includes a pedestal which is attached to the skull of the patient such as with glues and/or bone screws, preferably in the same surgical procedure in which sensor 200 is implanted in the brain of patient 500. Electronic module 170 attaches to transcutaneous connector 165 via threads, bayonet lock, magnetic coupling, velcro, or other engagement means. Transcutaneous connector 165 and/or electronic module 170 may include integrated electronics including but not limited to signal amplifier circuitry, signal filtration circuitry, signal multiplexing circuitry, and other signal processing circuitry, such that transcutaneous connector 165 and/or electronic module 170 provide at least a portion of the processing unit of the disclosed invention. Transcutaneous connector 165 preferably includes electrostatic discharge protection circuitry. Electronic module 170 includes wireless information transfer circuitry, utilizing one or more of radiofrequency, infrared, ultrasound, microwave, or other wireless communication means. In an alternative embodiment, transcutaneous connector 165 includes all the appropriate electronic signal processing, electrostatic discharge protection circuitry, and other circuitry, and also includes wireless transmission means, such that the need for electronic module 170 is obviated.

In a preferred embodiment, electronic module 170 includes wireless transmission means and a power supply, not shown, such that, as the power supply is depleted or electronic module 170 has a malfunction, it can be easily replaced. In another preferred embodiment, electronic module 170 is a disposable component of system 100″. Electronic module 170 transmits information to processing unit transceiver 135 which is integrated into a portion of system 100″s processing unit, such as processing unit first portion 130a. In a preferred embodiment, processing unit transceiver 135 is a two-way wireless communication device, and electronic module 170 is also a two-way wireless communication device such that information can be sent to or from electronic module 170.

All of the physical cables of FIG. 4, as well as all the other figures of this disclosure, can be in a permanently attached, or in a detachable form. In addition, all of the physical cables included in system 100″ of FIG. 4 as well as the systems of the other included figures can be eliminated with the inclusion of wireless transceiver means incorporated into the applicable, communicating discrete components. Processing unit first portion 130a, a discrete component as defined in this disclosure, includes various signal processing functions as has been described in detail in relation to separate figures hereabove. Processing unit first portion 130a preferably includes a unique system identifier, the makeup and applicability of the unique identifier also described in detail hereabove. Processing unit first portion 130a electrically connects to processing unit second portion 130b via intra-processing unit cable 140. Cable 140 is detachable from processing unit second portion 130b via female plug 153 which is attached to processing unit second portion 130b at its input port, male receptacle 152. Cable 140 may be constructed of electrical wires and/or fiber optic cables. In a preferred embodiment, data is transmitted from processing unit first portion 130a to processing unit second portion 130b via a fiber optic cable. Information and other signals transmitted between processing unit first portion 130a and processing unit second portion 130b may be in analog format, digital format, or a combination of both. In addition, wireless transmission of information can be provided, not shown, to replace intraprocessing unit cable 140 or work in conjunction with intraprocessing unit cable 140.

Processing unit second portion 130b includes further signal processing means which in combination with the signal processing of processing unit first portion 130a produces processed signals, such as to control multiple controlled devices. Processing unit first portion 130a and/or processing unit second portion 130b include various functions including but not limited to: a spike sorting function, such as a threshold based neuron spike sorting function; an amplifier function; a signal filtering function; a neural net software function; a mathematical signal combination function; a neuron signal separation function such as a spike discrimination function or a minimum amplitude sorting function; and a database storage and retrieval function such as a database including a list of acceptable neural information or a database of unacceptable neural information each of which can be used to perform a system diagnostic. In another preferred embodiment, the processing unit assigns one or more cellular signals to a specific use, such as a specific use that is correlated to a patient imagined event.

The processed signals emanating from processed unit second portion 130b can be analog signals, digital signals, or a combination of analog and digital signals. The processing unit of the present invention may include digital to analog conversion means as well as analog to digital conversion means. The processed signals can be transmitted to one or more controlled devices with a hardwired connection, a wireless connection or a combination of both technologies. As depicted in FIG. 4, controlled computer 305, first controlled device 300a, and second controlled device 300b are controlled by the processed signals produced by processing unit first portion 130a and processing unit second portion 130b. Similar to processing unit first portion 130a, processing unit second portion 130b preferably includes the system unique electronic identifier, which can be embedded in processing unit second portion 130b at the time of manufacture, during installation procedures, during calibration or other post-surgical configuration procedures, or at a later date.

The three controlled devices are shown permanently attached to physical cables, with each physical cable including a removable connection at the other end. Controlled computer 305 is attached to cable 311 that has female plug 155 at its end. First controlled device 300a is attached to first controlled device cable 301a which has female plug 159 at its end. Second controlled device 300b is attached to second controlled device cable 301b which has female plug 157 at its end. Each physical cable can be attached and detached from processing unit second portion 130b. Female plug 159 attaches to male receptacle 158; female plug 157 attaches to male receptacle 156, and female plug 155 attaches to male receptacle 154.

Each of controlled computer 305, first controlled device 300a, and second controlled device 300b preferably has embedded within it a unique identifier of the particular device. Additional codes, such as the unique system identifier, may also be embedded. When any of the physical cables are first attached, such as controlled computer cable 311 being attached via female plug 157 to male receptacle 156, a compatibility check is performed by system 100″ to assure that the unique system identifier embedded in controlled computer 305 is identical or otherwise compatible with a unique electronic identifier embedded in any and all other discrete components of system 100″, such as the unique electronic identifier embedded in processing unit second portion 130b. Similar system compatibility checks can be performed with the attachment of first controlled device 300a or second controlled device 300b. If improper compatibility is determined by system 100″, various actions that can be taken include but are not limited to: entering an alarm state, displaying incompatibility information, transmitting incompatibility information, deactivation of controlled device control, limiting controlled device control, and other actions.

Also depicted in FIG. 4 is selector module 400 which can be used by the patient or a different operator, such as a clinician, to select one or more specific devices to be controlled by the processed signals of system 100″. Selector module 400 includes numerous elements and functional capability as has been described in detail in relation to FIG. 1. Selector module 400 is shown with a data entry keypad, input element 402, and an output element 403, such as a visual display. Input element 402 is used by an operator to select the specific controlled device, and to perform other data entry. Output element 403 provides information to the operator such as selectable controlled device icons, controlled device information, and other system information. Selector module 400 communicates with processing unit first portion 130a via wireless technology, information transfer means 410. After selection of the one or more controlled devices to be controlled by the processed signals, these processed signals include one or more unique codes identifying the selected controlled device or devices, and may additionally include the unique system identifier. These codes can be sent at the initiation or cessation of control or on a periodic or continuous basis in order to assure that only the selected devices are controlled by the processed signals. A selection event can either cause a controlled device to begin to be controlled or stop the control of a controlled device that is already being controlled. In a preferred embodiment, specific operators can select specific equipment, such conditional matrix stored in a memory module of selector module 400 or other discrete component of system 100″.

Selector module 400 may include access passwords or require mechanical or electronic keys to prevent unauthorized use, and may also include a function, such as a permission routine function, to select a controlled device to modify its control. Selector module 400 may have other integrated functions such as information recall functions, system configuration, or calibration functions, as well as a calculator, cellular telephone, pager, or personal data assistant (PDA) functions. Clinician control unit 400 may be a PDA that has been modified to access system 100″ to select one or more controlled device to modify its control, such as through the use of a permission routine.

Selector module 400 of FIG. 4 includes an integrated monitor for displaying the information, however in an alternative embodiment, the selector module 400 can cause the information to be displayed on a separate visualization apparatus such as the monitor of controlled computer 305. Alternatively or additionally, one or more of the functions of the selector module 400 can be integrated into one or more discrete components of system 100″.

Numerous configurations and types of controlled devices can be used with system 100″ of FIG. 4. Numerous types of controlled devices have been described in detail in relation to the systems of FIG. 1 and FIG. 3 and are applicable to system 100″ of FIG. 4 as well. System 100″ works with a single patient 500 who can control multiple controlled devices such as controlled computer 305, first controlled device 300a, and second controlled device 300b. In a preferred embodiment, patient 500 can select and/or control more than one controlled device simultaneously. While each controlled device is connected to the same discrete component, such as processing unit second portion 130b, in an alternative embodiment, the multiple controlled devices can be connected to multiple processing unit discrete components. In that embodiment, the selector module 400 is used to start or stop the transmission of the individual processing units to their corresponding controlled device.

While patient 500 has been implanted with a sensor 200 including a single discrete component, sensor 200 may comprise multiple discrete components, not shown, such as multiple electrode arrays, implanted in different parts of the brain, or in other various patient locations to detect multicellular signals. Cellular signals from the individual sensor discrete components, such as a single electrode component, may be sent to individual processing units, or to a single processing unit. Separate processed signals can be created from each individual discrete component of the sensor, and those particular signals tied to a specific controlled device. Thus, each controlled device can be controlled by processed signals from a different sensor discrete assembly, such as discrete components at different locations in the brain or other parts of the body. It should be appreciated that any combination of discrete component cellular signals can be used in any combination of multiple controlled devices. Alternatively, whether the sensor is embodied in a single discrete component or multiple discrete components, the processed signals for individual controlled devices may be based on specific cellular signals or signals from specific electrodes, such that individual device control is driven by specific cellular signals. Any combination of exclusively assigned cellular signals and shared cellular signals used to create processed signals for multiple controlled devices are to be considered within the scope of this application. In an alternative, preferred embodiment, the system includes multiple patients, these patients collectively selecting and/or controlling one or more controlled devices.

The system 100″ of FIG. 4 may include two or more separate configuration routines, such as a separate calibration routine for each controlled device. Any and all discrete components of system 100″ may have a unique electronic identifier embedded in it. The processing unit of system 100″, comprising processing unit first portion 130a and processing unit second portion 130b, may conduct adaptive processing as has been described hereabove.

The unique electronic identifier of the system is a unique code used to differentiate one system, such as the system of a single patient, from another system, as well as to differentiate all discrete components of a system, especially detachable components, from discrete components of a separate, potentially incompatible system. The unique electronic identifier may be a random alphanumeric code or may include information including but not limited to: patient name, other patient information, system information, implant information, number of electrodes implanted, implant location or locations, software revisions of one or more discrete components, clinician name, date of implant, date of calibration, calibration information, manufacturing codes, and hospital name. In a preferred embodiment, the unique electronic identifier is stored in more than one discrete component such as a sensor discrete component and a processing unit discrete component. The unique electronic identifier may be programmable, such as one time programmable, or allow modifications for multiple time programming, such programming performed in the manufacturing of the particular discrete component, or by a user at a later date. The unique electronic identifier may be configured to be changed over time, such as after a calibration procedure. The unique electronic identifier can be permanent or semi-permanent, or hard wired, such as a hard wired configuration in a transcutaneous connector of the system. The unique electronic identifier can be used in wireless communications between discrete components, or in wireless communications between one or more discrete components and a device outside of the system. The unique electronic identifier can represent or be linked to system status. System status can include but not be limited to: output signal characteristics, level of accuracy of output signal, output signal requirements, level of control needed, patient login settings, such as customized computer configuration information, one or more software revisions, one or more hardware revisions, controlled device compatibility list, patient permissions lists, and calibration status. In a preferred embodiment, the unique identifier includes information to identify the system as a whole, as well as information identifying each discrete component, such as each controlled device applicable to the system. The unique portion identifying each controlled device can be used in wireless communication, after a selection has been made via the selector module, such that the selected controlled devices are properly controlled.

The system 100″ of FIG. 4 may include a library of various integrated parameters, such integrated parameters utilized by the processing units, processing unit first portion 130a and processing unit second portion 130b to perform a function including but not limited to the creation of the processed signals to control one or more controlled devices. Integrated parameters include various pieces of system data, such as data stored in electronic memory. In a preferred embodiment, the data being electronically linked with the unique electronic identifier of system 100″. The integrated parameter data may be stored in memory of one or more discrete components, such as processing unit second portion 130b, or alternatively or additionally the integrated parameter data may be stored in a computer based network platform, separate from system 100′ such as a local area network (LAN), a wide area network (WAN) or the Internet. The integrated parameter data can contain numerous categories of information related to the system including but not limited to: patient information such as patient name and disease state; discrete component information such as type of sensor and electrode configuration; system configuration information such as calibration dates, calibration output parameters, calibration input parameters, patient training data, signal processing methods, algorithms and associated variables, controlled device information such as controlled device use parameters and lists of controlled devices configured for use with or otherwise compatible with the system; and other system parameters useful in using, configuring and assuring safe and efficacious performance of system 100″.

In an alternative embodiment, system 100″ of FIG. 4 further comprises a patient feedback module. The feedback module may include one or more of an audio transducer, a tactile transducer, and a visual display. This patient feedback module may be used during patient training, or at all times that the patient is controlling an external device. Feedback can be used to enhance external device control as well as to avoid unsafe or undesirable conditions. The feedback module may utilize one or more discrete components of system 100″ such as sensor 200. In another preferred embodiment, one or more electrodes of sensor 200 can be stimulated, such as via a stimulation circuit provided by one or more of transcutaneous connector 165 or electronic module 170. The stimulation can evoke a variety of responses including but not limited to the twitching of a patient's finger. The feedback signal sent to the patient can take on a variety of forms, but is preferably a derivative of a modulating variable of the controlled device. For example, feedback can be a derivative of cursor position of controlled computer 305. If audio feedback is implemented, a signal representing horizontal position and a signal representing vertical position can be combined and sent to a standard speaker. Other audio feedback, such as specific discrete sounds, can be incorporated to represent proximity to an icon, etc. Parameters of the feedback module should be considered integrated parameters of the systems of this invention, such that one or more feedback parameters require approval of an operator via the system's permission routine. In a preferred embodiment, the patient feedback function is incorporated into selector module 400 such as via a visual display or audio transducer.

Patient 500 of FIG. 4 is at a specific location, Location 1. An operator such as a clinician operator 111 is at a location remote from patient 500, Location 2. Also at Location 2 is configuration system 120 which can access system 100″ via the Internet as has been described in reference to previous embodiments. Configuration system 120 can be used to perform various configuration procedures such as calibration procedures as has been described in reference to a similar configuration system of FIG. 3. In a preferred embodiment, configuration system 120 can perform the functions of the selector module such that clinician operator 111 can select a specific device to modify its control via configuration apparatus 120 and the Internet.

Numerous methods are provided in the multiple embodiments of the disclosed invention. A preferred method embodiment includes a method of selecting a specific device to be controlled by the processed signals of a biological interface system. The method comprises: providing a biological interface system for collecting multicellular signals emanating from one or more living cells of a patient and for transmitting processed signals to control a device. The biological interface system comprises: a sensor for detecting the multicellular signals, the sensor comprising a plurality of electrodes to allow for detection of the multicellular signals; a processing unit for receiving the multicellular signals from the sensor, for processing the multicellular signals to produce processed signals, and for transmitting the processed signals; a first controlled device for receiving the processed signals; a second controlled device for receiving the processed signals; and a selector module that is used to select the specific device to be controlled by the processed signals.

It should be understood that numerous other configurations of the systems, devices, and methods described herein can be employed without departing from the spirit or scope of this application. It should be understood that the system includes multiple functional components, such as a sensor for detecting multicellular signals, a processing unit for processing the multicellular signals to produce processed signals, and the controlled device that is controlled by the processed signals. Different from the logical components are physical or discrete components, which may include a portion of a logical component, an entire logical component, and combinations of portions of logical components and entire logical components. These discrete components may communicate or transfer information to or from each other, or communicate with devices outside the system. In each system, physical wires, such as electrical wires or optical fibers, can be used to transfer information between discrete components, or wireless communication means can be utilized. Each physical cable can be permanently attached to a discrete component, or can include attachment means to allow attachment and potentially allow, but not necessarily permit, detachment. Physical cables can be permanently attached at one end, and include attachment means at the other.

The sensors of the systems of this application can take various forms, including multiple discrete component forms, such as multiple penetrating arrays that can be placed at different locations within the body of a patient. The processing unit of the systems of this application can also be contained in a single discrete component or multiple discrete components, such as a system with one portion of the processing unit implanted in the patient, and a separate portion of the processing unit external to the body of the patient. The sensors and other system components may be utilized for short term applications, such as applications less than twenty four hours, sub-chronic applications such as applications less than thirty days, and chronic applications. Processing units may include various signal conditioning elements such as amplifiers, filters, signal multiplexing circuitry, signal transformation circuitry, and numerous other signal processing elements. In a preferred embodiment, an integrated spike sorting function is included. The processing units perform various signal processing functions including but not limited to: amplification, filtering, sorting, conditioning, translating, interpreting, encoding, decoding, combining, extracting, sampling, multiplexing, analog to digital converting, digital to analog converting, mathematically transforming and/or otherwise processing cellular signals to generate a control signal for transmission to a controllable device. Numerous algorithms and/or mathematical and software techniques can be utilized by the processing unit to create the desired control signal. The processing unit may utilize neural net software routines to map cellular signals into desired device control signals. Individual cellular signals may be assigned to a specific use in the system. The specific use may be determined by having the patient attempt an imagined movement or other imagined state. For most applications, it is preferred that that the cellular signals be under the voluntary control of the patient. The processing unit may mathematically combine various cellular signals to create a processed signal for device control.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims. In addition, where this application has listed the steps of a method or procedure in a specific order, it may be possible, or even expedient in certain circumstances, to change the order in which some steps are performed, and it is intended that the particular steps of the method or procedure claim set forth herebelow not be construed as being order-specific unless such order specificity is expressly stated in the claim.

What is claimed is:

1. A biological interface system comprising:
a sensor comprising a plurality of electrodes configured to detect multicellular signals emanating from one or more living cells of a patient, the sensor being implanted within the skull of the patient;
a processing unit configured to receive the multicellular signals from the sensor, to process the multicellular signals to produce processed signals, and to transmit the processed signals; and
a controlled device configured to receive the processed signals from the processing unit;
wherein the processing unit comprises a processing unit first portion and a processing unit second portion, and
wherein the processing unit first portion is implanted under the scalp on the skull of the patient, and the processing unit second portion is placed above the scalp of the patient at a location proximal to the processing unit first portion.

2. The system of claim 1, wherein the processing unit first portion is connected to the sensor with a bundle of electrically conductive wires.

3. The system of claim 2, wherein the wire bundle comprises a separate wire attached to each electrode.

4. The system of claim 1, wherein the processing unit first portion lacks an integrated supply of power.

5. The system of claim 1, wherein the processing unit first portion comprises an inductive coil.

6. The system of claim 5, wherein the inductive coil is configured to convert received electromagnetic signals to power.

7. The system of claim 5, wherein the inductive coil is configured to convert received electromagnetic signals to data.

8. The system of claim 1, wherein the processing unit first portion is configured to transmit information via an infrared communication element.

9. The system of claim 8, wherein the infrared communication element comprises an infrared light emitting diode.

10. The system of claim 9, wherein the processing unit first portion comprises one or more optical components to focus the emitted infrared light.

11. The system of claim 1, wherein the processing unit first portion comprises a signal processing circuitry.

12. The system of claim 11, wherein the signal processing circuitry is configured to perform one or more of: amplification; filtering; sorting; conditioning; translating; interpreting; encoding; decoding; combining; extracting; sampling; multiplexing; analog to digital converting; digital to analog converting; mathematically transforming; and processing cellular signals to generate a control signal for transmission to a controllable device.

13. The system of claim 1, wherein the processing unit first portion comprises an integrated power supply.

14. The system of claim 1, wherein the processing unit first portion comprises one or more of: a temperature sensor; a pressure sensor; a strain gauge; an accelerometer; a volume sensor; an electrode; an array of electrodes; an audio transducer; a mechanical vibrator; a drug delivery device; a magnetic field generator; a photo detector element; a visualization apparatus; a wireless communication element; a light producing element; an electrical stimulator; a physiologic sensor; a heating element; and a cooling element.

15. The system of claim 1, wherein the processing unit first portion is configured to transmit information to the processing unit second portion with a wireless information transfer member.

16. The system of claim 15, wherein the processing unit second portion is external to the patient.

17. The system of claim 1, wherein the processing unit first portion is placed on the skull in close proximity to the ear of the patient.

18. The system of claim 1, wherein the processing unit first portion is placed in a recess surgically created on the top of the skull of the patient.

19. The system of claim 1, wherein the processing unit second portion comprises an integrated power supply.

20. The system of claim 1, wherein the processing unit second portion is configured to transmit the processed signals to the controlled device via wireless communication.

21. The system of claim 1, wherein the processing unit second portion comprises an infrared receiving element.

22. The system of claim 21, wherein the infrared receiving element comprises a photodiode.

23. The system of claim 21, wherein the infrared receiving element comprises one or more optical components used to collect the infrared light.

24. The system of claim 1, wherein the processing unit second portion comprises an inductive coil.

25. The system of claim 24, wherein the inductive coil is configured to send power to the processing unit first portion.

26. The system of claim 25, wherein the inductive coil is configured to send data to the processing unit first portion.

27. The system of claim 1, wherein the controlled device comprises a first controlled device and a second controlled device.

28. The system of claim 27, further comprising a selector module configured to select which of the first and second controlled devices is to be controlled by the processed signals.

29. The system of claim 1, wherein the system comprises a neural interface system.

30. The system of claim 1, wherein the system comprises a brain machine interface.

31. The system of claim 1, wherein the system is configured to perform a therapeutic function.

32. The system of claim 31, wherein the therapeutic function comprises a treatment of one or more of: obesity, an eating disorder, a neurological disorder, a psychiatric disorder, a cardiovascular disorder, an endocrine disorder, sexual dysfunction, incontinence, a hearing disorder, a visual disorder, a sleeping disorder, a movement disorder, a speech disorder, physical injury, migraine headaches, and chronic pain.

33. The system of claim 1, wherein the system is configured to perform a patient diagnosis.

34. The system of claim 33, wherein the patient diagnosis comprises a diagnosis of one or more of: obesity, an eating disorder, a neurological disorder, a psychiatric disorder, a cardiovascular disorder, an endocrine disorder, sexual dysfunction, incontinence, a hearing disorder, a visual disorder, sleeping disorder, a movement disorder, a speech disorder, physical injury, migraine headaches, and chronic pain.

35. The system of claim 1, wherein the system is configured to restore a bodily function of the patient.

36. The system of claim 35, wherein the bodily function of the patient comprises one or more of vision, hearing, speech, communication, limb motion, ambulation, reaching, grasping, standing, rolling over, bowel movement, and bladder evacuation.

37. The system of claim 1, wherein the system is configured to be turned on and off by the patient with a monitored biological signal.

38. The system of claim 37, wherein the monitored biological signal is generated by one or more of eye motion, eyelid motion, facial muscle, or other electromyographic activity.

39. The system of claim 37, wherein the monitored biological signal comprises a time code of brain activity.

40. The system of claim 1, wherein the multicellular signals emanate from the central nervous system of the patient.

41. The system of claim 1, wherein the multicellular signals emanate from a single cell of the patient.

42. The system of claim 1, wherein the multicellular signals comprise one or more of: neuron spikes, electrocorticogram signals, local field potential signals, and electroencephalogram signals.

43. The system of claim 1, wherein the electrodes are configured to detect the multicellular signals from clusters of neurons and provide signals between single neuron and electroencephalogram recordings.

44. The system of claim 1, wherein the processing unit is configured to assign at least one cellular signal to a specific use.

45. The system of claim 1, wherein the processing unit is configured to use at least one cellular signal generated under voluntary control of a patient.

46. The system of claim 1, wherein the patient is a human being.

47. The system of claim 1, wherein the patient is one or more of: a quadriplegic, a paraplegic, an amputee, a spinal chord injury victim, and a physically impaired person.

48. The system of claim 1, wherein the patient is healthy, and the system is not configured to provide a therapeutic or restorative function to the patient.

49. The system of claim 48, wherein the controlled device comprises a medical equipment.

50. The system of claim 49, wherein the medical equipment is configured to perform a surgical event.

51. The system of claim 48, wherein the controlled device comprises a communication device.

52. The system of claim 51, wherein the communication device is configured to transmit different pieces of information simultaneously.

53. The system of claim 48, wherein the controlled device comprises a piece of equipment with one or more controllable moving parts.

54. The system of claim 53, wherein the equipment is used to evacuate personnel.

55. The system of claim 53, wherein the equipment is used to diffuse a bomb.

56. The system of claim 53, wherein the equipment is used to provide a military function.

57. The system of claim 53, wherein the equipment is one or more of a: watercraft, aircraft, land vehicle, and reconnaissance robot.

58. The system of claim 1, wherein the controlled device comprises one or more of the group consisting of: a computer, a computer display, a mouse, a cursor, a joystick, a personal data assistant, a robot or robotic component, a computer controlled device, a teleoperated device, a communication device, a vehicle, an adjustable bed, an adjustable chair, a remote controlled device, a Functional Electrical Stimulator device, a muscle stimulator, an exoskeletal robot brace, an artificial or prosthetic limb, a vision enhancing device, a vision restoring device, a hearing enhancing device, a hearing restoring device, a movement assist device, a medical therapeutic equipment, a drug delivery apparatus, a medical diagnostic equipment, a bladder control device, a bowel control device, a human enhancement device, and a closed loop medical equipment.

59. The system of claim 1, wherein the sensor comprises at least one multi-electrode array comprising the plurality of electrodes.

60. The system of claim 59, wherein the plurality electrodes are arranged in a ten by ten array.

61. The system of claim 59, wherein the plurality of electrodes are configured to penetrate into neural tissue of the brain to detect electric signals generated from neurons.

62. The system of claim 59, wherein the multi-electrode array comprises at least one of a recording electrode, a stimulating electrode, and an electrode having recording and stimulating capabilities.

63. The system of claim 59, wherein the multi-electrode array comprises multiple projections extending from a surface, and at least one of the multiple projections comprises at least one electrode along its length.

64. The system of claim 63, wherein at least one of the multiple projections includes no electrode.

65. The system of claim 63, wherein at least one of the multiple projections includes an anchoring member.

66. The system of claim 59, wherein the sensor further comprises a second multi-electrode array.

67. The system of claim 1, wherein the sensor comprises multiple wires or wire bundle electrodes.

68. The system of claim 1, wherein the plurality of electrodes of the sensor are incorporated into one or more of: a subdural grid, a scalp electrode, a wire electrode, and a cuff electrode.

69. The system of claim 1, wherein the electrodes comprise wires, and the sensor comprises a wire bundle.

70. The system of claim 1, wherein the sensor comprises two or more discrete components.

71. The system of claim 70, wherein each of the discrete components comprises one or more electrodes.

72. The system of claim 70, wherein each of the discrete components comprises one or more of: a multi-electrode array; a wire or wire bundle; a subdural grid; and a scalp electrode.

73. The system of claim 1, wherein the electrodes are configured to detect multicellular signals for less than twenty-four hours.

74. The system of claim 1, wherein the electrodes are configured to chronically detect multicellular signals.

75. The system of claim 1, wherein the sensor further comprises a signal processing circuitry.

76. The system of claim 1, wherein the sensor transmits the multicellular signals through a wireless connection.

77. The system of claim 76, wherein the sensor transmits wirelessly to a receiver mounted on the skull of the patient.

78. The system of claim 1, wherein the sensor further comprises a coil for power transmission to the sensor.

79. The system of claim 1, wherein the plurality of electrodes are configured to record from clusters of neurons and output detected signals, the detected signals comprising multiple neuron signals.

80. The system of claim 79, wherein the detected signals are a measure of the local field potential response from neural activity.

81. The system of claim 79, wherein the multiple neuron signals comprise one or more of: electrocorticogram signals, local field potentials, electroencephalogram signals, and peripheral nerve signals.

82. The system of claim 1, wherein one or more of the plurality of electrodes is configured to detect a plurality of neuron signals.

83. The system of claim 1, wherein a portion of the processing unit is physically connected to the sensor.

84. The system of claim 1, wherein the processing unit comprises an integrated neuron spike sorting function.

85. The system of claim 84, wherein the neuron spike sorting function classifies spikes with a minimum amplitude threshold.

86. The system of claim 1, wherein the processing unit comprises an element to amplify the multicellular signals.

87. The system of claim 86, wherein the signals are amplified by a gain of at least eighty.

88. The system of claim 1, wherein the processing unit utilizes neural net software routines to map neural signals into the processed signals for control of the controlled device.

89. The system of claim 44, wherein the specific use is determined by the patient attempting an imagined movement or state.

90. The system of claim 1, wherein the processing unit is configured to utilize two or more cellular signals that are mathematically combined to create the processed signals.

91. The system of claim 1, wherein the processing unit is configured to use a cellular signal from a neuron whose signal is separated from other nearby neurons.

92. The system of claim 91, wherein the processing unit is configured to separate signals by one or more spike discrimination methods.

93. The system of claim 92, wherein the spike discrimination method sorts spikes by a minimum amplitude threshold.

94. The system of claim 1, wherein the processing unit is configured to convert an analog signal that represents a cellular signal to a digital signal.

95. The system of claim 94, wherein the processing unit is configured to process a monitored biological signal and produce a second processed signal.

96. The system of claim 95, wherein the second processed signal is used to control the controlled device.

97. The system of claim 95, wherein the second processed signal is used to modify one or more parameters of the system.

98. The system of claim 95, wherein the second processed signal is used to stop control of the controlled device.

99. The system of claim 95, wherein the second processed signal is used to reset the system.

100. The system of claim 94, further comprising a permission routine, wherein the permission routine requires approval of the operator, and wherein one or more integrated parameters of the system are modified.

101. The system of claim 100, wherein the permission routine limits parameter modifications to one or more specific operators.

102. The system of claim 101, wherein the permission routine comprises a list of approved operators.

103. The system of claim 101, wherein permission to modify individual integrated parameters is linked to one or more specific operators.

104. The system of claim 101, wherein a specific operator is permitted to approve modification of a parameter within a range of values.

105. The system of claim 104, wherein the range of values is controlled by a second operator.

106. The system of claim 100, wherein the permission routine comprises multiple levels including permissions for multiple operators.

107. The system of claim 106, wherein a first operator controls a first set of one or more integrated parameters, and a second operator controls a second set of one or more integrated parameters.

108. The system of claim 107, wherein the first set of parameters comprises one or more different parameters than the second set of parameters.

109. The system of claim 100, further comprising an interrogation function which interrogates the system and retrieves information stored therein.

110. The system of claim 109, wherein an analysis is performed on the retrieved information, and an output is produced, the output comprising a recommendation for modifying at least one of the integrated parameters.

111. The system of claim 100, wherein prior to implementing a modification, the permission routine checks one or more of: username, password, and IP address.

112. The system of claim 100, wherein the permission routine comprises a confirmation of modifications prior to implementing a modification.

113. The system of claim 1, further comprising an adaptive processing routine.

114. The system of claim 113, wherein the adaptive processing routine comprises changing over time the type or combination of types of signals processed.

115. The system of claim 114, wherein the types of signals processed comprise one or more of EEG signals, ECoG signals, LFP signals, and neural spikes.

116. The system of claim 1, further comprising a configuration system for calibrating the multicellular signals.

117. The system of claim 116, wherein the configuration system is configured to be activated by a biological signal.

118. The system of claim 116, wherein the configuration system comprises a set of movements for configuration.

119. The system of claim 116, wherein the configuration system comprises a video monitor.

120. The system of claim 119, wherein the configuration system comprises a set of movements for configuration, and the video monitor is configured to display a selected movement.

121. The system of claim 120, wherein the movements displayed are from the patient's perspective.

122. The system of claim 116, wherein the configuration system is configured to correlate the selected movement with a cellular signal obtained from tracking the selected movement.

123. The system of claim 116, wherein the configuration system is configured to correlate an integrated parameter relating to the selected movement with a cellular signal obtained from tracking the selected movement.

124. The system of claim 123, wherein the integrated parameter comprises one or more of a position, a velocity, or an acceleration.

125. The system of claim 119, wherein the configuration system comprises a set of movements for configuration, and the video monitor is configured to display a simulation of a selected movement.

126. The system of claim 125, wherein the simulation of the selected movement is displayed from the patient's perspective.

127. The system of claim 1, further comprising a patient feedback module.

128. The system of claim 127, wherein the patient feedback module comprises one or more of: an audio transducer, a tactile transducer, a visual transducer, a video display, and an olfactory transducer.

129. The system of claim 127, wherein the patient feedback module comprises a stimulator, and one or more neurons are stimulated to cause movement or sensation in a part of the patient's body.

130. The system of claim 1, further comprising a drug delivery system, wherein the processing unit transmits a signal to the drug delivery system to deliver a therapeutic agent or drug to at least a portion of the patient's body.

131. The system of claim 1, further comprising an embedded identification.

132. The system of claim 131, wherein the embedded identification is used to confirm compatibility of one or more discrete components of the system.

133. A method comprising:
providing a biological interface system for collecting multicellular signals emanating from one or more living cells of a patient and for transmitting processed signals to control a device, the biological interface system comprising:
a sensor comprising a plurality of electrodes configured to detect the multicellular signals; and
a processing unit configured to receive the multicellular signals from the sensor and to process the multicellular signals to produce processed signals;
cutting a hole into the skull of the patient;
inserting the sensor through the hole into a portion of the brain;
creating a recess in the skull;
placing the processing unit in the recess;
detecting the multicellular signals using the sensor;
processing the detected multicellular signals to produce processed signals;
transmitting the processed signals via wireless communication through the scalp.

134. The method of claim 133, wherein the wireless communication comprises infrared communication.

135. The method of claim 133, wherein the processing unit comprises a processing unit first portion placed in the recess of the skull and a processing unit second portion placed above the scalp of the patient, and wherein transmitting the processed signals comprises transmitting the processed signals from the processing unit first portion to the processing unit second portion.

136. The method of claim 135, further comprising transmitting the processed signals from the processing unit second portion to the device to be controlled.

137. The method of claim 136, wherein transmitting the processed signals from the processing unit second portion to the device to be controlled is performed via wireless communication.

138. The method of claim 133, wherein the processing unit comprises a processing unit first portion placed in the recess of the skull and a processing unit second portion placed above the scalp of the patient, and wherein the method further comprises transmitting power from the processing unit second portion to the processing unit first portion.

139. The method of claim 138, wherein each of the processing unit first portion and the processing unit second portion comprises a coil assembly, and wherein transmitting power is achieved by inductive coupling between the coil assembly of the processing unit first portion and the coil assembly of the processing unit second portion.

140. The method of claim 139, wherein the transmitting power comprises supplying a driving signal to the coil assembly of the processing unit second portion to generate an electromagnetic field that, through inductive coupling, generates power in the coil assembly of the processing unit first portion.

141. The method of claim 133, wherein the processing unit comprises a processing unit first portion placed in the recess of the skull and a processing unit second portion placed above the scalp of the patient, and wherein the method further comprises transmitting information from the processing unit second portion to the processing unit first portion.

142. The method of claim 141, wherein each of the processing unit first portion and the processing unit second portion comprises a coil assembly, and wherein transmitting information comprises transmitting information between the coil assembly of the processing unit first portion and the coil assembly of the processing unit second portion.

143. The method of claim 142, wherein the transmitting information comprises modulating waveform with circuitry of the coil assembly of the processing unit second portion to transmit information.

144. The method of claim 143, wherein the transmitting information comprises receiving and decoding the transmitted information by the coil assembly of the processing unit first portion.
